(19) United States
(12) Patent Application Publication
(10) Pub. No.: US 2007/0060512 A1
(43) Pub. Date: Mar. 15, 2007

(54) Dipeptidyl-peptidase protected protein

(76) Inventors: Homayoun Sadeghi, King of Prussia, PA (US); Christopher P. Prior, King of Prussia, PA (US); David J. Ballance, King of Prussia, PA (US)

Correspondence Address: COOLEY GODWARD KRONISH LLP;ATTN: Patent Group, Suite 500, 1200 - 19th Street, NW, WASHINGTON, DC, 20036-2402, US

(21) Appl. No.: 10/547,510

(22) PCT Filed: Mar. 4, 2004
(86) PCT No.: PCT/US04/06462

§ 371 Date: Aug. 31, 2006

(30) Foreign Application Priority Data
Mar. 4, 2003 (US) 10378094
Aug. 28, 2003 (US) 03/26818

(51) Int. Cl. A61K 38/26; A61K 38/08

(52) U.S. Cl. 514/12; 514/18; 530/399; 530/330

(57) ABSTRACT

The present invention provides modified therapeutic polypeptides or peptides partially or completely protected from DPP activity. The modified polypeptides or peptides comprise at least one additional amino acid at the amino terminus. The modified therapeutic polypeptides or peptides are useful in the treatment of diseases such as diabetes.

### RELATED APPLICATION

This application is a Continuation-in-Part of PCT/US03/26818, filed Aug. 28, 2003, which is a Continuation-in-Part of U.S. application Ser. No. 10/378,094, filed Mar. 4, 2003, both of which are herein incorporated by reference in their entirety.

### FIELD OF THE INVENTION

This invention relates to modified polypeptides that are resistant to dipeptidyl peptidase cleavage. Specifically, this invention includes modified insulinotropic peptides that are protected from dipeptidyl peptidase IV. The methods of the invention include extending the effective therapeutic in vivo half life of the modified insulinotropic peptides.

### BACKGROUND OF THE INVENTION

Proteases

Proteolytic enzymes play an important role in regulating physiological processes such as cell proliferation, differentiation, and signaling processes by regulating protein turnover and processing. Proteolytic enzyme controls the levels of important structural proteins, enzymes, and regulatory proteins through proteolytic degradation. Uncontrolled proteolytic enzyme activity, either increased or decreased, has been implicated in a variety of disease conditions including inflammation, cancer, arteriosclerosis, and degenerative disorders.

The International Union of Biochemistry and Molecular Biology (IUBMB) has recommended the use of the term “peptidase” for the subset of peptide bond hydrolases (Subclass E.C 3.4.). The widely used term protease is synonymous with peptidase. Peptidases comprise two groups of enzymes: the endopeptidases and the exopeptidases, which cleave peptide bonds at points within the protein and remove amino acids sequentially from either N or C-terminus respectively. The term proteinase is synonymous with endopeptidase. Proteolytic enzymes are classified according to their catalytic mechanisms. Four mechanistic classes have been recognized by the IUBMB: the serine proteases, cysteine proteases, aspartic proteases, and metalloproteases.

Serine proteases are a large family of proteolytic enzymes containing a serine residue in the active catalytic site for protein cleavage. They are ubiquitous being found in viruses, bacteria, and eukaryotes. Serine proteases have a wide range of substrate specificities and can be subdivided into subfamilies on the basis of these specificities. There are over 20 subfamilies of serine proteases which are grouped into six clans (SA, SB, SC, SE, SF, and SG).

Prolyl oligopeptidase is a serine protease grouped in the SC clan. It hydrolyzes proline containing peptides at the carboxyl side of proline residues. Presumably, it is involved in the maturation and degradation of peptide hormones and neuropeptides (Wilk et al. 1983 Life Sci. 33, 2149-2157). Examples of prolyl oligopeptidase include dipeptidyl peptidase IV (DPPIV), dipeptidyl peptidase II (DPPII), fibroblast activation protein, and prolyl oligopeptidase. These enzymes display distinct specificities.

Proline is present in numerous peptide hormones. It determines certain structural properties of these peptides, such as conformation and stability of these peptides, preventing degradation by non-specific proteases. A number of peptidases exist which attack the proline bonds. These peptidases are not only involved in the cleavage of X-Pro or Pro-X bonds, but also in the degradation of corresponding alanyl bonds, with reduced activity. Peptidases having highly specific actions on proline-containing sequences are attractive targets of medicinal chemistry because some of them have been linked to the modulation of the biological activity of natural peptide substrates. For example, DPPIV is linked to the treatment of diabetes through regulating the level of glucagon-like peptide-1 (GLP-1). DPPIV activity is increased in various diseases such as rheumatoid arthritis, multiple sclerosis, Grave's disease, and Hashimoto's thyroiditis, sarcoidosis, and cancer. DPPIV activity is also increased in AIDS, Down's syndrome, anorexia/bulimia, pregnancy and hypogammaglobulinemia.

Dipeptidyl Peptidases Including DPPIV

Dipeptidyl aminopeptidase activity is peptidase activity which catalyzes the removal of dipeptides from the N-terminus of peptides, polypeptides, and proteins. Generally, a dipeptidyl aminopeptidase is capable of cleaving the dipeptide XY from the unsubstituted N-terminal amino group of a peptide, polypeptide or protein, wherein X and Y represent any amino acid residue. Examples of dipeptidyl peptidases (DPPs) include dipeptidyl peptidase I (DPPI), dipeptidyl peptidase II (DPPII), dipeptidyl peptidase III (DPPIII), and dipeptidyl peptidase (DPPIV).

DPPI, also known as cathepsin C, is a lysosomal cysteine protease that is expressed in most tissues. DPPI has been implicated in the processing of granzymes, which are neutral serine proteases expressed exclusively in the granules of activated cytotoxic lymphocytes. DPPII is a serine protease found in lysosomes. Like DPPIV, it cleaves proline containing peptide bonds. In fact, DPPII has a similar substrate specificity to DPPIV but is only active at acidic pH. Dipeptidyl peptidase III (DPPIII) is a metalloprotease.

DPPIV is a serine protease comprising the serine protease motif GWSYG and having broad substrate specificity. It hydrolyzes a peptide in sequence from the amino terminus to release an amino acid. However, the hydrolysis is terminated when an amino acid residue followed by proline is reached. As a result, a peptide having a bond of X-Pro-Y- (X and Y are optional amino acids) will be cleaved to yield X-Pro and Y-. DPPIV will also cleave dipeptides with alanine in the penultimate position, though less effectively than dipeptides with proline (Yaron et al., 1993 Crit. Rev. Biochem. Mol. Biol. 28:31-81). The enzyme will also cleave other sequences, but with still lower efficiency.

DPPIV has been shown to be highly specific in releasing dipeptides from the N-terminal end of biologically active peptides with proline or alanine in the penultimate position of the N-terminal sequence of the peptide substrate. A large number of potential peptide substrates for DPPIV have been identified. DPPIV substrates include peptide hormones and chemokines. Examples of some peptide hormones are endomorphin-2, GLP-1, GLP-2, gastric inhibitory peptide (GIP), neuropeptide Y, growth hormone releasing hormone (GHRH) and substance P, and examples of some chemokines are RANTES, GCP-2, SDF-1α, SDF-2β, MDC, MCP-1, MCP-2, and MCP-3. DPPII possesses almost identical substrate specificity to DPPIV.

DPPIV and Diabetes

Insulin-dependent diabetes mellitus (IDDM, or type I diabetes) is currently treated through the administration of insulin to patients. Non-insulin-dependent diabetes mellitus (NIDDM, or type II diabetes) is treated by diet, administration of sulphonylureas to stimulate insulin secretion or with biguanides to increase glucose uptake. Resistant individuals may need insulin therapy. Standard therapy requires daily intravenous injection of insulin which will treat the acute symptoms, but prolonged therapy results in vascular disease and nerve damage. Modern methods such as transplantation are expensive and require risky surgical intervention. Thus, there is a need to develop a highly effective, low cost alternative to the treatment of diabetes.

In recent years, there has been a growing interest in DPPIV as a target for lowering the level of blood glucose. The use of inhibitors to block DPPIV enzyme or DPPIV-like enzyme activity in the blood of subjects leads to reduced degradation of endogenous or exogenously administered insulinotropic peptides such as, GIP, GLP-1 or analogs thereof. GIP and GLP-1, hormones that stimulate glucose-induced secretion of insulin by the pancreas, are substrates of DPPIV. Specifically, since DPPIV removes the amino-terminal His-Ala dipeptide of GLP-1 to generate GLP-1-(9-36)-amide, which is unable to elicit glucose-dependent insulin secretion from the islets, the inhibition of such DPPIV or DPPIV-like enzyme activity in vivo would effectively suppress undesired enzyme activity in pathological conditions in mammalian organisms.

PCT/DE97/00820 discloses alanyl pyrrolidide and isoleucyl thiazolidide as inhibitors of DPPIV or DPPIV-like enzyme activity. DD 296075 discloses pyrrolidide and isoleucyl thiazolidide hydrochloride. U.S. Pat. No. 6,548,481 discloses inhibitors analogous to dipeptide compounds formed from an amino acid and a thiazolidine or pyrrolidine group, and salts thereof. Although these are functional inhibitors of DPPIV activities, the use of these inhibitors in certain patients or certain forms of the disease may be problematic since the enzyme is responsible for activation or inactivation of such a wide range of bioactive peptides, i.e. DPPIV inhibitors lack specificity for the desired targets GIP and GLP-1.

Protection of Therapeutic Peptides by Modification

An alternative way to prevent therapeutic proteins and peptides such as GIP or GLP-1 from being cleaved by proteolytic enzymes is to modify the proteins and peptides themselves to block their exposure to proteolytic enzymes. Protein modifications have been shown to increase therapeutic polypeptides' stability, circulation time, and biological activity. Some general methods of modifying amino acids and peptides are disclosed in Chemistry and Biochemistry of Amino Acids, Peptides, and Proteins—A Survey of Recent Developments (Weinstein, B., ed., Marcel Dekker, Inc., publ., New York 1983) which is incorporated herein by reference. Also, the review article of Francis (1992 Focus on Growth Factors 3:4-10, (Mediscript, London)) describes protein modification and fusion proteins, which is incorporated herein by reference.

With the advance of recombinant DNA technology and automated techniques, one may now easily prepare large quantities of modified polypeptides that are short, medium or long. A large number of modified small polypeptide hormones may be synthesized using automated peptide synthesizers, solid-state resin techniques, or recombinant techniques. For example, large quantities of modified substrates of dipeptidyl peptidase, for example, the substrates of DPPIV such as GLP-1, GIP, neuropeptide Y, and bradykinin can be produced using an automated peptide synthesizer.

### SUMMARY OF THE INVENTION

The present invention provides modified therapeutic peptides and proteins that are resistant to dipeptidyl protease cleavage. The inventors discovered that modifying the amino terminus of dipeptidyl peptidase substrates by adding at least one additional N-terminal amino acid protects these peptide substrates from dipeptidyl peptidase activity. Specifically, the inventors found that adding one or more amino acids to the amino terminus of the peptide substrates of DPPIV blocks DPPIV and DPPIV-like protease activity. Such modified substrates have enhanced biological stability in the blood of mammals and would be more effective as a therapeutic peptides or proteins. As an example, GLP-1 is a substrate of DPPIV activity. Modified GLP-1 peptide that are protected from DPPIV cleavage are more stable and more effective in lowering elevated blood glucose levels in mammals.

The present invention provides modified therapeutic polypeptides and peptides that contain one to five additional amino acids at its N-terminus. The modified polypeptides and peptides are partially or substantially resistant to DPP cleavage. The modification reduces DPP cleavage activity by about 10%, about 30%, about 50%, about 70%, or about 90% as compared to the polypeptide prior to modification. The modified polypeptide or peptide has retained about 10%, about 30%, about 50%, about 70%, and about 90% of its activity and/or potency as compared to the polypeptide prior to modification.

The present invention also provides modification of variants of therapeutic polypeptides or peptides that may have an increased or decreased functional activity as compared to their respective wild-type therapeutic polypeptide or peptide. Moreover, the present invention provides fusion proteins comprising modified polypeptides or peptides linked to a second protein, for example transferrin or albumin, for increased stability.

Specifically, the present invention provides modified GLP-1 comprising one or more additional amino acids at its N-terminus. The present invention also provides fusion protein comprising modified GLP-1 resistant to DPP cleavage and transferrin. The GLP-1 peptide may be the wild-type peptide or a variant or analog thereof. The modified GLP-1 peptide may be fused to conjugated to a heterologous molecule such as a polyethylene glycol, a fatty acid, or fatty acid derivative.

In one embodiment, the present invention includes nucleic acids encoding the modified polypeptides or peptides. In another embodiment, the invention provides vectors and host cells comprising the nucleic acids encoding the modified polypeptides or peptides. The present invention also disclose the use of the nucleic acid constructs for expression in vivo, for example in a mammal.

The modified polypeptides and peptides of the present invention are useful for treating diseases. Specifically, the modified GLP-1 peptides are useful for treating diseases or conditions associated with abnormal blood glucose level. The modified GLP-1 peptides of the present invention are used to treat subjects with diabetes and obesity. The subjects may be mammals. The mammals may be humans.

### BRIEF DESCRIPTION OF THE DRAWINGS

FIG. 1 shows the restriction enzyme map of pREX0094.

FIG. 2 shows the restriction enzyme map of plasmid pREX0198.

FIG. 3 shows the restriction enzyme map of pSAC35.

FIG. 4 shows the restriction enzyme map of plasmid pREX0240.

FIG. 5 shows the restriction enzyme map of pREX0052.

FIG. 6 shows the restriction enzyme map of pREX0367.

FIG. 7 shows the restriction enzyme map of pREX0368.

FIG. 8 shows time course of incubation of GLP-1 and H-GLP-1 and DPP-IV. The graph shows the amount of active, full length peptide remaining, as measured by an ELISA specific for active GLP-1.

### DETAILED DESCRIPTION

1. General Description

This invention is based, in part, on the need to develop a more effective, low cost alternative for the treatment of diabetes. Insulinotropic peptides, such as GLP-1, are promising therapeutic agents for the treatment of type 2 non-insulin-dependent diabetes mellitus as well as related metabolic disorders, such as obesity. Other useful insulinotropic peptides include exendin 3 and exendin 4. However, these insulinotropic peptides have short plasma half-lifes in vivo, mainly due to rapid serum clearance and proteolytic degradation. Extensive work has been done to inhibit DPPIV, the enzyme responsible for the degradation of GLP-1 or to modify GLP-1 in such a way that its degradation is slowed down while still maintaining biological activity. Despite these extensive efforts, a long lasting, active GLP-1 has not been produced. There is thus a need to modify GLP-1, exendin 3, exendin 4 and other insulinotropic peptides to provide longer duration of action in vivo, while maintaining their low toxicity and therapeutic advantages.

The present invention is based in part on the finding that modification of a dipeptidyl peptidase (DPP) substrate, by adding one or more amino acids at the N-terminus of the substrate renders the substrate resistant to DPP cleavage while maintaining biological activity. Specifically, the inventors discovered that adding one or more amino acids to GLP-1 substantially protects GLP-1 from DPPIV enzyme activity. The modified GLP-1 of the present invention may be useful in the treatment of diseases associated with abnormal level of blood glucose, such as diabetes.

Accordingly, the present invention provides modification of the substrates of dipeptidyl peptidases including but not limited to DPPII, DPPIV, and prolyl oligopeptidase. The addition of one or more amino acids to the amino terminus of these substrates protects them from dipeptidyl peptidase cleavage. Thus, these modified substrates are more stable.

2. Definitions

As used herein, the term “derivative” refers to a modification of one or more amino acid residues of a peptide by chemical means, either with or without an enzyme, e.g., by alkylation, acylation, ester formation, or amide formation.

As used herein, the term “derived from” refers to obtaining a molecule from a specified source such as obtaining a molecule from a parent molecule.

As used herein, the term “dipeptidyl aminopeptidase activity” refers to a peptidase activity which cleaves dipeptides from the N-terminal end of a peptide, polypeptide, or protein sequence. Generally, the dipeptidyl aminopeptidase is capable of cleaving the dipeptide XY from the unsubstituted N-terminal amino group of a peptide, polypeptide, or protein, wherein X or Y may represent any amino acid residue selected from the group consisting of Ala, Arg, Asn, Asp, Cys, Gln, Glu, Gly, His, Ile, Leu, Lys, Met, Phe, Pro, Ser, Thr, Trp, Tyr, and Val, but at least Ala, Arg, Asp, and/or Gly. All of X and Y may be different or identical. Examples of dipeptidyl aminopeptidase include, but are not limited to DPPI, DPPII, DPPII, and DPPIV.

As used herein, the terms “Glucagon-Like Peptide-1 (GLP-1)” and “GLP-1 derivatives” refer to intestinal hormones which generally simulate insulin secretion during hyperglycemia, suppresses glucagon secretion, stimulates (pro) insulin biosynthesis and decelerates gastric emptying and acid secretion. Some GLP-1s and GLP-1 derivatives promote glucose uptake by cells but do not simulate insulin expression as disclosed in U.S. Pat. No. 5,574,008 which is hereby incorporated by reference.

As used herein, the term “insulinotropic peptides” refers to peptides with insulinotropic activity. Insulinotropic peptides stimulate, or cause the stimulation of, the synthesis or expression of the hormone insulin. Such peptides include precursors, analogues, fragments of peptides such as Glucagon-like peptide, exendin 3 and exendin 4 and other peptides with insulinotropic activity.

As used herein, “pharmaceutically acceptable” refers to materials and compositions that are physiologically tolerable and do not typically produce an allergic or similar untoward reaction, such as gastric upset, dizziness and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutical composition” refers to a composition comprising an agent together with a pharmaceutically acceptable carrier or diluent when needed. Pharmaceutically acceptable carriers and additives are chosen such that side effects from the pharmaceutical compound are minimized and the performance of the compound is not canceled or inhibited to such an extent that treatment is ineffective.

As used herein, “physiologically effective amount” is that amount delivered to a subject to give the desired palliative or curative effect. This amount is specific for each drug and its ultimate approved dosage level.

As used herein, “therapeutically effective amount” refers to that amount of modified therapeutic polypeptide or peptide which, when administered to a subject in need thereof, is sufficient to effect treatment. The amount of modified therapeutic polypeptide or peptide which constitutes a “therapeutically effective amount” will vary depending on the therapeutic protein used, the severity of the condition or disease, and the age and body weight of the subject to be treated, but can be determined routinely by one of ordinary skill in the art having regard to his/her own knowledge and to this disclosure.

As used herein, “therapeutic protein” refers to proteins, polypeptides, antibodies, peptide fragments or variants thereof, having one or more therapeutic and/or biological activities. Therapeutic proteins encompassed by the invention include but are not limited to proteins, polypeptides, peptides, antibodies and biologics. The terms peptides, proteins, and polypeptides are used interchangeably herein. Additionally, the term “therapeutic protein” may refer to the endogenous or naturally occurring correlate of a therapeutic protein. By a polypeptide or peptide displaying a “therapeutic activity” or a protein that is “therapeutically active” is meant a polypeptide, peptide or protein that possesses one or more known biological and/or therapeutic activities associated with a therapeutic protein such as one or more of the therapeutic proteins described herein or otherwise known in the art. As a non-limiting example, a “therapeutic protein” is a protein, polypeptide, or peptide that is useful to treat, prevent or ameliorate a disease, condition or disorder. Such a disease, condition or disorder may be in humans or in a non-human animal, e.g., veterinary use.

As used herein, the term “treatment” or “treating” refers to any administration of a compound of the present invention and includes: (1) preventing the disease from occurring in an animal which may be predisposed to the disease but does not yet experience or display the pathology or symptomatology of the disease; (2) inhibiting the disease in an animal that is experiencing or displaying the pathology or symptomatology of the diseased (i.e., arresting further development of the pathology and/or symptomatology); or (3) ameliorating the disease in an animal that is experiencing or displaying the pathology or symptomatology of the diseased (i.e., reversing the pathology and/or symptomatology).

3. Specific Embodiments

Dipeptidyl Peptidases

Dipeptidyl peptidases are hydrolases that remove dipeptides from the unsubstituted N-terminal amino group of a peptide, polypeptide, or protein. Examples of dipeptidyl peptidases include but are not limited to DPPI, DPPII, DPPIII, DPPIV, attractin, and fibroblast activation protein (FAP). New enzymes of this family or with similar function but different structure are emerging.

Dipeptidyl peptidase I (DPPI), also known as cathepsin C, is a lysosomal cysteine protease belonging to the papain family. DPPI is capable of sequentially removing dipeptides from the free amino terminus of various peptide and protein substrates, thus acting in the exopeptidase (specifically dipeptidyl peptidase) mode. The cleavage is ineffective if the fragmented bond has on either side a proline residue, or the N-terminal residue is lysine or arginine.

DPPII is a serine protease found in lysosomes with unknown function. Like DPPIV, it cleaves proline containing peptide bonds. In fact, DPPII has a similar substrate specificity as DPPIV but is only active at acidic pH. Mammalian DPPII and DPPIV can be distinguished using the inhibitors puromycin and bacitracin; puromycin will inhibit DPPII only while bacitracin inhibits DPPIV only (1988 J. Biol. Chem. 263, 6613-6618). Dipeptidyl peptidase III (DPPIII) is a metalloprotease. DPPV releases N-terminal X-Ala, His-Ser, and Ser-Tyr dipeptides.

DPP-VII, also known as quiescent cell proline dipeptidase, is a proline-specific dipeptidases. It has been suggested that DPPVII and DPPII are identical proteases based on a sequence comparison of human DPP-VII and rat DPP-II (78% identity) (Araki et al. 2001 J. Biochem. 129, 279-288).

DPPVIII is a human postproline dipeptidyl aminopeptidase that is homologous to DPPIV and FAP (Abbott, C. A. et al., 2000 European Journal of Biochemistry 267, 6140). Similar to DPPIV, DPPVIII is ubiquitous. The full-length DPPVIII cDNA codes for an 882-amino-acid protein that has about 27% identity and 51% similarity to DPPIV and FAP, but no transmembrane domain and no N-linked or O-linked glycosylation. Purified recombinant DPPVIII hydrolyzed the DPPIV substrates Ala-Pro, Arg-Pro and Gly-Pro. Thus recombinant DPPVIII shares a postproline dipeptidyl aminopeptidase activity with DPPIV and FAP. DPPVIII enzyme activity had a neutral pH optimum consistent with it being nonlysosomal. The similarities between DPPVIII and DPPIV in tissue expression pattern and substrates suggests a potential role for DPPVIII in T-cell activation and immune function similar to DPPIV.

Olsen C. et al. (2002 Gene 299, 185-93) report the identification and characterization of a novel reports a novel DPP IV-like molecule, termed dipeptidyl peptidase-like protein DPPIX. Like DPPIV, DPPIX comprises the serine protease motif GWSYG (SEQ ID NO: 110). The presence of this motif and the conserved order and spacing of the Ser, Asp, and His residues that form the catalytic triad in DPPIV, places DPPIX in the DPPIV gene family.

Attractin (DPPT-L) is a 175-kDa soluble glycoprotein reported to hydrolyze Gly-Pro. Attractin contains a kelch repeat domain and shares no significant sequence homology with DPPIV or any other peptidase. Fibroblast activation protein (FAP) is a cell surface-bound protease of the prolyl oligopeptidase gene family expressed at sites of tissue remodelling.

Prolyl endopeptidase (PEP), also called proline oligopeptidase (PO), was first discovered by Walter and coworkers as an oxytocin-degrading enzyme in the human uterus (Walter et al., Science 173, 827-829 (1971)). The enzyme cleaves peptide bonds at the carboxy-side of proline in peptides containing the sequence X-Pro-Y, where X is a peptide or N-terminal substituted amino-acid and Y is a peptide, amino acid, amide or alcohol (Yoshimoto et al., J. Biol. Chem. 253, 3708-3716 (1979)). The enzyme has a high specificity for the trans-conformation of the peptide bond at the imino-side of proline (Lin & Brandts, Biochemistry 22, 4480-4485 (1983)).

Prolyl oligopeptidase hydrolyzes angiotensin I and angiotensin II which results in the relase of angiotensin (1-7). Angiotensin (1-7) has vasodilator activity and modulates the release of vasopressin, which is able to influence the process of memory as was shown by injecting rats with specific PEP-inhibitors. The injection reverses the scopolamine induced amnesia. This experiment is not only an example which provides evidence for a possible physiologic function for the enzyme, but moreover it has led to the hypothesis that inhibitors for PEP can influence the memory process and counter dementia (Yoshimoto et al. 1987 J. Pharmacobio-Dyn. 10, 730-735).

Dipeptidyl Peptidase (DPPIV) and Substrates

DPPIV is a ubiquitously expressed molecule that has been implicated in the degradation of several peptides and hormones. Various types of DPPIV have been purified and the enzymological properties have been revealed. For example, DPPIV has been isolated from rat liver (Hopsu-Havu V. K. et al., 1966 Histochem., 7:197-201), swine kidney (Barth A. et al., 1974 Biol. Med. Chem., 32:157-174), small intestine (Svensson B. 1978 Eur. J. Biochem., 90:489-498), liver (Fukasawa K. M. et al. 1981 Biochim. Biophys. Acta, 657:179-189), human submaxillary gland (Oya H., et al., 1972 Biochim. Biophys. Acta, 258:591-599), sheep kidney (Yoshimoto T. et al., 1977 Biochim. Biophys. Acta, 485:391-401; Yoshimoto T. et al., 1978 J. Biol. Chem., 253:3708-3716) or microorganisms (Fukusawa K. M. 1981 Biochem. Biophys., 210:230-237; Yoshimoto T. 1982 J. Biochem., 91:1899-1906 (1982)).

In the human immune system, DPPIV is identical to the T-cell surface antigen CD26 which is expressed by activated lymphocytes (T-, B-, and natural killer cells). CD26/DPPIV is a Type II membrane glycoprotein with intrinsic dipeptidyl peptidase IV activity and the ability to bind adenosine deaminase Type I (ADA-1). It is expressed on epithelial cells constitutively, but on T lymphocytes, it is expressed under tight cellular regulation, with expression upregulated upon cell activation. CD26/DPPIV has been shown to have dipeptidyl peptidase IV activity in its extracellular domain (Hegen et al., 1990 J. Immunol 144:2908-2914; Ulmer et al., 1990 Scand. J. Immunol. 31:429-435) and the costimulatory activity appears to be partially dependent upon this enzyme activity (Tanaka et al., 1993 Proc. Natl. Acad. Sci. USA 90:4586-4590). DPPIV is involved in the regulation of chemokine function and may play an important role in HIV infection.

U.S. Pat. No. 6,265,551 discloses a circulating, soluble form of DPPIV/CD26 isolated from human serum. The serum form shares similar enzymatic and antigenic properties with the ubiquitous membrane form; however, in several biochemical aspects there are distinct differences. In particular, the circulating serum form has a molecular weight of 175 kDa, in contrast to the 105 kDa molecular weight of the membrane form, and it does not bind ADA-1. Nevertheless, the circulating form expresses functional dipeptidylpeptidase IV activity and retains the ability to costimulate the T lymphocyte response to recall antigen.

The proteolytic activity of DPPIV resides in a stretch of approximately 200 amino acids located at the C-terminal end of the protein. The catalytic residues (Ser-629, Asp-708, His-740) are arranged in a unique order which is different from the classical serine proteases such as chymotrypsin and subtilisin. Proline specific dipeptidyl peptidase activity alters the biological activity of a large number of bioactive proteins and polypeptides comprising, amongst others, GLP-1, the neurotransmitter substance P, human growth hormone-releasing factor, erythropoietin, interleukin 2 and many others. Potential DPPIV substrates are listed in Tables 1, 2 and 3. Modulation of these polypeptides to affect DPPIV cleavage may be useful in the treatment of clinical conditions including but not limited to diabetes, inflammation, vascular diseases, auto-immune disease, multiple sclerosis, joint diseases and diseases associated with benign and malign cell transformation.

TABLE 1

Human cytokines, growth factors, neuro- and
vasoactive peptides with a penultimate proline,
which are putative substrates for DPP IV

| | | |
|---|---|---|
| | SEQ | |
| | ID | |
| Polypeptide | NO: | N-terminal sequence |

| | | |
|---|---|---|
| Interleukin-1.beta. | 1 | Ala-Pro-Val-Arg-Ser- |
| Interleukin-2 | 2 | Ala-Pro-Thr-Ser-Ser- |
| Interleukin-5 | 3 | Ile-Pro-Thr-Glu-Ile- |
| Interleukin-6 | 4 | Val-Pro-Pro-Gly-Glu- |
| Interleukin-10 | 5 | Ser-Pro-Gly-GIn-Gly- |
| Interleukin-13 (recombinant) | 6 | Ser-Pro-Gly-Pro-Val- |
| Complement C4a | 7 | Lys-Pro-Arg-Leu-Leu- |
| Granulocyte chemotactic protein II | 8 | Gly-Pro-Val-Ser-Ala- |
| Granulocyte macrophage colony stimulating | 9 | Ala-Pro-Ala-Arg-Ser- |
| Factor | | |
| Granulocyte colony stimulating factor | 10 | Thr-Pro-Leu-Gly-Pro- |
| Erythopoietin | 11 | Ala-Pro-Pro-Arg-Leu- |
| Gastrin releasing peptide growth hormone | 12 | Phe-Pro-Thr-Ile-Pro- |
| Interferon inducible peptide 10 (.gamma.IP10) | 13 | Val-Pro-Leu-Ser-Arg- |
| Interferon regulatory factor 1 (IRF-1) | 14 | Met-Pro-Ile-Thr-Arg |
| Interferon regulatory factor 2 (IRF-2) | 15 | Met-Pro-Val-Glu-Arg |
| Insulin-like growth factor-1 | 16 | Gly-Pro-Glu-Thr-Leu- |
| Melanoma growth stimulating activity | 17 | Ala-Pro-Leu-Ala-Thr- |
| Migration inhibition factor | 18 | Met-Pro-Met-Phe-Ile- |
| Monocyte chemotactic protein I | 19 | Glu-Pro-Asp-Ala-Ile- |
| Neuropeptide Y | 20 | Tyr-Pro-Ser-Lys-Pro- |
| Pancreatic polypeptide | 21 | Ala-Pro-Leu-Glu-Pro- |
| Peptide YY | 22 | Try-Pro-Ile-Lys-Pro- |
| Prolactin | 23 | Leu-Pro-Ile-Cys-Pro- |
| RANTES | 24 | Ser-Pro-Tyr-Ser-Ser- |
| Substance P | 25 | Arg-Pro-Lys-Pro-Gln- |
| Thrombopoietin | 26 | Ser-Pro-Ala-Pro-Pro- |
| Transforming protein (N-myc) version 1 | 27 | Met-Pro-Gly-Met-Ile- |
| Transforming protein (N-myc) version 2 | 28 | Met-Pro-Ser-Cys-Ser- |
| Tumor necrosis factor.beta. | 29 | Leu-Pro-Gly-Val-Leu- |
| Vascular endothelial growth factor | 30 | Ala-Pro-Met-Ala-Glu |

TABLE 2
Human peptides and proteins with a penultimate alanine
that are putative substrates for DPP IV

adenosine deaminase
Annexins
breast basic conserved protein
Cofilin
natural killer cell enhancing factor b
precursors of α-interferon
precursors of interleukin 1, α and 1, β and interleukin 13
precursors of macrophage inflammatory protein-2-α and 2-β
precursor of melanocyte stimulating hormone
precursor of oxytocin-neurophysin 1
growth hormone releasing hormone
β amyloid protein (1-28)
anxiety peptide
joining peptide of pro-opiomelanocortin

The present invention provides modified substrates of DPP comprising one or more additional amino acids at the N-terminus of the substrates to protect the substrates from DPP activity. The preferred substrates for modification according to the present invention are disclosed in Table 3.

TABLE 3
Substrates for DPP-IV (CD26) Cleavage

| | SEQ | |
|---|---|---|
| | ID | |
| DPP-IV Substrate | NO: | Sequence |
| GIP | 31 | YAEGTFISDY SIAMDKIHQQ DFVNWLLAQK |
| | | GKKNDWKHNI TQ |
| GLP-1 | 32 | HAEGTFTSDV SSYLEGQAAK EFIAWLVKG |
| | (Amino | |
| | Acids | |
| | 1-29) | |
| GLP-2 | 33 | HADGSFSDEM NTILDNLAAR DFINWLIQTK ITD |
| growth hormone | 34 | YADAIFTNSY RKVLGQLSAR KLLQDIMSRQ |
| releasing hormone | | QGESNQERGA RARL |
| Glucagon (slow | 35 | HSQGTFTSDY SKYLDSRRAQ DFVQWLMNT |
| inactivation, unlike | | |
| GIP and the GLPs) | | |
| peptide histidine- | 36 | HADGVFTSDF SKLLGQLSAK KYLESLM |
| methionine | | |
| IGF-1 | 37 | G PETLCGAELV DALQFVCGDR GFYFNKPTGY GSSSPRAPQT |
| | | GIVDECCFRS CDLRRLEMYC APLKPAKSAR SVRAQRHTDM |
| | | PKAQKEVHLK NASRGSAGNK TY |
| Bradykinin | 38 | RPPGFSPFR |
| Substance P | 39 | RPKPQQFFGL M |
| CLIP | 40 | RPVKVYPNGA EDESAEAFPL EF |
| Neuropeptide Y | 41 | YPSKPDNFGE DAPAEDMARY YSALRHYINL ITRQRY |
| peptide YY (DPPIV | 42 | YPIKPEAPGE DASPEELNRY YASLRHYLNL VTRQRY |
| activates it) | | |
| Prolactin | 43 | LPICPGGAA RCQVTLRDLF DRAVVLSHYI HNLSSEMFSE |
| | | FDKRYTHGRG FITKAINSCH TSSLATPEDK EQAQQMNQKD |
| | | FLSLIVSILR SWNEPLYHLV TEVRGMQEAP EAILSKAVEI |
| | | EEQTKRLLEG MELIVSQVHP ETKENEIYPV WSGLPSLQMA |
| | | DEESRLSAYY NLLHCLRRDS HKIDNYLKLL KCRIIHNNNC |
| human chorionic | 44 | (alpha subunit) APDVQDCPEC TLQEDPFFSQ PGAPILQCMG |
| gonadotropin (HCG) | | CCFSRAYPTP LRSKKTMLVQ KNVTSESTCC VAKSYNRVTV |
| | | MGGFKVEDHT ACHCSTCYYH KS |
| human chorionic | 45 | (beta subunit) SKEPLRPRCR PINATLAVEK EGCPVCITVN |
| gonadotropin (HCG) | | TTICAGYCPT MTRVLQGVLP ALPQVVCNYR NVRFESIRLP |
| | | GCPRGVNPVV SYAVALSCQC ALCRRSTTDC GGPKDHPLTC |
| | | DDPRFQDSSS SKAPPPSLPS PSRLPKPSDT PILPQ |
| enterostatin | 46 | APGPR |
| gastrin-releasing | 47 | VPLPAGGGTV LTKMYPRGNH WAVGHLM |
| peptide | | |
| IL-2 | 48 | APTSSSTKKT QLQLEHLLLD LQMILNGINN YKNPKLTRML |
| | | TFKFYMPKKA TELKHLQCLE EELKPLEEVL NLAQSKNFHL |
| | | RPRDLISNIN VIVLELKGSE TTFMCEYADE TATIVEFLNR |
| | | WITFCQSIIS TLT |
| IL-1b | 49 | APVR SLNCTLRDSQ QKSLVMSGPY ELKALHLQGQ |
| | | DMEQQVVFSM SFVQGEESND KIPVALGLKE KNLYLSCVLK |
| | | DDKPTLQLES VDPKNYPKKK MEKRFVFNKI EINNKLEFES |
| | | AQFPNWYIST SQAENMPVFL GGTKGGQDIT DFTMQFVSS |
| endomorphin-2 | 50 | YPFF |
| tyr-melanostatin | 51 | YPLG |
| aprotinin | 52 | RPDFCLEPPY TGPCKARIIR YFYNAKAGLC QTFVYGGCRA |
| | | KRNNFKSAED CMRTCGGA |
| RANTES | 53 | SPYSSDTTPC CFAYIARPLP RAHIKEYFYT SGKCSNPAVV |
| | | FVTRKNRQVC ANPEKKWVRE YINSLEMS |
| trypsinogen | 54 | NPLLILTFV AAALAAPFDD DDKIVGGYNC EENSVPYQVS |
| | | LNSGYHFCGG SLINEQWVVS AGHCYKSRIQ VRLGEHNIEV |
| | | LEGNEQFINA AKIIRHPQYD RKTLNNDIML IKLSSRAVIN |
| | | ARVSTISLPT APPATGTKCL ISGWGNTASS GADYPDELQC |
| | | LDAPVLSQAK CEASYPGKIT SNMFCVGFLE GGKDSCQGDS |
| | | GGPVVCNGQL QGVVSWGDGC AQKNKPGVYT |
| | | KVYNYVKWIK NTIAANS |
| alpha1-microglobulin | 55 | G PVPTPPDNIQ VQENFNISRI YGKWYNLAIG STCPWLKKIM |
| | | DRMTVSTLVL GEGATEAEIS MTSTRWRKGV CEETSGAYEK |
| | | TDTDGKFLYH KSKWNITMES YVVHTNYDEY AIFLTKKFSR |
| | | HHGPTITAKL YGRAPQLRET LLQDFRVVAQ GVGIPEDSIF |
| | | TMADRGECVP GEQEPEPILI PRV |
| interferon-inducible | 56 | VPLSRTVRCT CISISNQPVN PRSLEKLEII PASQFCPRVE |
| protein 10 (IP10) | | IIATMKKKGE KRCLNPESKA IKNLLKAVSK ERSKRSP |
| Eotaxin | 57 | GPASVPTTCC FNLANRKIPL QRLESYRRIT SGKCPQKAVI |
| | | FLTKLAKDIC ADPKKKYVQD SMKYLDQKSP TPKP |
| Monocyte | 58 | QPDAINAPVT CCYNFTNRKI SVQRLASYRR ITSSKCPKEA |
| chemoattractant | | VIFKTIVAKE ICADPKQKWV QDSMDHLDKQ TQTP |
| protein 1 (MCP-1) | | |
| Monocyte | 59 | QPDSVSIPIT CCFNVINRKI PIQRLESYTR ITNIQCPKEA |
| chemoattractant | | VIFKTKRGKE VCADPKERWV RDSMKHLDQI FQNLKP |
| protein 2 (MCP-2) | | |
| Monocyte | 60 | QPVGINTSTT CCYRFINKKI PKQRLESYRR TTSSHCPREA |
| chemoattractant | | VIFKTKLDKE ICADPTQKWV QDFMKHLDKK TQTPKL |
| protein 3 (MCP-3) | | |
| Granulocyte | 61 | GPV SAVLTELRCT CLRVTLRVNP KTIGKLQVFP |
| chemotactic | | AGPQCSKVEVV ASLKNGKQVC LDPEAPFLKK |
| protein-2 | | VIQKILDSGN KKN |
| SDF-1a | 62 | KPVSLSYRCP CRFFESHVAR ANVKHLKILN TPNCALQIVA |
| | | RLKNNNRQVC IDPKLKWIQE YLEKALNK |
| SDF-1b | 63 | KPVSLSYRCP CRFFESHVAR ANVKHLKILN TPNCALQIVA |
| | | RLKNNNRQVC IDPKLKWIQE YLEKALNKRF KM |
| Macrophage-derived | 64 | GPYGANMEDS VCCRDYVRYR LPLRVVKHFY |
| chemokine | | WTSDSCPRPG VVLLTFRDKE ICADPRVPWV KMILNKLSQ |
| b-casomorphin | 65 | YPFVEPI |
| Procolipase | 66 | APG PRGIIINLEN GELCMNSAQC KSNCCQHSSA |
| | | LGLARCTSMA SENSECSVKT LYGIYYKCPC ERGLTCEGDK |
| | | TIVGSITNTN FGICHDAGRS KQ |
| Vasoactive | 67 | HSDAVFTD - - |
| Intestinal | | |
| Peptide (VIP) | | |
| Pituitary Adenylyl | 68 | HSDGIF- - |
| Cyclase-Activating | | |
| Peptide 38 (PACAP38) | | |
| Oxyntomodulin | 69 | HSQGTFTS - - |
| Growth hormone (1-43) | 70 | FPTIPLSR - - |
| Secretin | 71 | HSDGTFTS - - |

The substrates for modification comprise X-ProY, X-Ala-Y, X-Ser-Y, or X-Gly-Y at the amino terminus. Preferably, the substrate for modification is GLP-1.

Modified Polypeptides Protected from DPP Activity

The present invention provides modified polypeptides, such as modified polypeptide substrates of DPP, comprising one or more additional amino acids at the N-terminus to protect the polypeptide substrates from DPP activity. In one embodiment, the modified polypeptides have one additional amino acid at its N-terminus as compared to their wild-type polypeptides. In another embodiment, the modified polypeptides have five additional amino acids at its N-terminus. Alternatively, the modified polypeptides have between one and five additional amino acids at its N-terminus. Any one of the 20 amino acids may be added to the N-terminus of the polypeptide substrate or non-natural amino acids may be added.

It is expected that any pharmaceutical polypeptide having peptide bonds which would be subject to cleavage in the gastrointestinal tract or anywhere in vivo after administration would benefit from modification in accordance with the present invention because of the protection from DPP cleavage that is afforded by the present invention.

In accordance with this aspect of the invention, it is possible to remove at least about 30%, preferably at least about 50%, more preferably at least about 70%, still more preferably at least about 90%, and most preferably at least about 99% of the dipeptidyl peptidase activity. It is also possible to completely remove the dipeptidyl aminopeptidase activity using the methods of the present invention.

Likewise, it is possible to reduce the substrate's dipeptidyl peptidase sensitivity by at least about 30%, preferably at least about 50%, more preferably at least about 70%, still more preferably at least about 90%, and most preferably at least about 99% of the dipeptidyl peptidase sensitivity. It is also possible to completely remove the dipeptidyl aminopeptidase sensitivity using the methods of the present invention.

Although the modified polypeptide or peptide substrates of the present invention are partially or substantially protected from DPP activity, the modified polypeptide substrates have retained at least about 30%, preferably at least about 50%, more preferably at least about 70%, and still more preferably at least about 90%, and most preferably at least about 99% of their functional activity and potency. In some instances, the modified polypeptide or peptide substrates with lowered functional activity or potency will be useful. For example, when the the modified polypeptide or peptide is fused to another polypeptide, such as transferrin, to form a fusion protein with increased serum stability and in vivo circulatory half-life, a modified polypeptide peptide substate with lowered functional activity or potency may be useful.

In other instances, the modified polypeptides or peptides have may increased potency as compared to the non-modified polypeptides or peptides.

Modified polypeptide molecules of the invention are substantially protected from dipeptidyl peptidase cleavage as compared to an unmodified version of the same polypeptide. Qualification of this substantial protection may vary by the assay used to compare the modified versus unmodified polypeptide. In order to exhibit substantial protection, however, the modified polypeptide will exhibit a detectable level of resistance to dipeptidyl peptidase cleavage in the assay. Such assays include but are not limited to those disclosed in Doyle et al. (2002 Endocrinology 142, 4462-4468), O'Harte et al. (1999 Diabetes 48, 758-765) and Siegel et al. (1999 Regulatory Peptides 79, 93-102).

DPP stabilized polypeptide substrates of the present invention are also more stable in the presence of DPP in vivo than a non-stabilized polypeptide substrates. A DPP stabilized therapeutic polypeptide substrate generally has an increased activity half-life as compared to a non-stabilized peptide of identical sequence. Peptidase stability may be determined by comparing the half-life of the unmodified polypeptide substrate in serum or blood to the half-life of a modified counterpart therapeutic peptide in serum or blood. Half-life may be determined by sampling the serum or blood after administration of the modified and non-modified peptides and determining the activity of the peptide. In addition to determining the activity, the length of the polypeptide substrates may also be measured by HPLC or Mass Spectrometry.

The present invention also provides modified polypeptides or peptides having an altered amino terminus according to the invention to protect against DPP cleavage and having internal and/or C-terminus amino acid alterations that do not affect the functional activity or potency of the polypeptide. These modified polypeptides would have minor amino acid changes that are usually conservative amino acid substitutions, although non-conservative substitutions are also contemplated.

The modified polypeptides or peptides of the present invention may also have altered functional activity. For instance, a modified polypeptide or peptide with increased functional activity may be useful. Alternatively, a modified polypeptide or peptide with decreased functional activity may be used. Thus, the modified polypeptides or peptides of the present invention also contain amino acid changes that do affect functional activity or potency. For example, the analogs of GLP-1 with altered functional activity may be modified at its amino terminus to protect against DPP cleavage.

Examples of conservative amino acid substitutions are substitutions made within the same group such as within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, valine), aromatic amino acids (such as phenylalanine, tryptophan, tyrosine) and small amino acids (such as glycine, alanine, serine, threonine, methionine).

Non-conservative substitutions encompass substitutions of amino acids in one group by amino acids in another group. For example, a non-conservative substitution would include the substitution of a polar amino acid for a hydrophobic amino acid. For a general description of nucleotide substitution, see e.g. Ford et al. (1991), Prot. Exp. Pur. 2: 95-107.

The present invention provides obvious variants of the amino acid sequence of the modified polypeptides and peptides, such as naturally occurring mature forms of the polypeptides or peptides, allelic/sequence variants of the polypeptides, non-naturally occurring recombinantly derived variants of the peptides, and orthologs and paralogs of the polypeptides or peptides. Such variants can readily be generated using art-known techniques in the fields of recombinant nucleic acid technology and protein biochemistry. Such variants can readily be identified/made using molecular techniques and the sequence information. Further, such variants can readily be distinguished from other peptides based on sequence and/or structural homology to the modified polypeptides or peptides of the present invention.

Preferably, the modified peptides of the present invention are GLP-1 and analogs thereof comprising one or more additional amino acids at their N-terminus.

In some instances, the DPP such as DPPIV may activate a peptide instead of inactivating it through cleavage. In such instances, modification of the peptide could substantially reduce, delay, or prevent peptide activation.

Nucleic Acids Encoding Modified Polypeptides

The present invention provides nucleic acid molecules encoding modified polypeptides and peptides that are partially or substantially protected from DPP cleavage and have functional activity and potency. In one embodiment, nucleic acid molecules provided by the present invention encode modified polypeptides and peptides having at least one additional amino acid at its N-terminus as compared to their wild-type unmodified polypeptide. In another embodiment, the nucleic acid molecules encode modified polypeptides and peptides having five additional amino acids at their N-terminus. Alternatively, the nucleic acid molecules encode modified polypeptides and peptides having between one and five additional amino acids at their N-terminus. Preferably, the nucleic acid molecules encoding modified GLP-1 comprise sequence encoding one or more additional amino acids at its N-terminus.

The nucleic acid molecules of the invention include deoxyribonucleic acids (DNAs), both single- and double-stranded deoxyribonucleic acids. However, they can also be ribonucleic acids (RNAs), as well as hybrid RNA:DNA double-stranded molecules. Contemplated nucleic acid molecules also include genomic DNA, cDNA, mRNA, and antisense molecules. The nucleic acids molecules of the present invention also include native or synthetic RNA, DNA, or cDNA that encode a modified polypeptide, or the complementary strand thereof.

To construct modified polypeptides that are partially or substantially protected from DPP activity but having functional activity and/or potency compared to wild-type unmodified polypeptides, the nucleic acid encoding the wild-type unmodified polypeptide can be used as a starting point and modified to encode the desired modified polypeptide. Numerous methods are known to add sequences or to mutate nucleic acid sequences that encode a polypeptide and to confirm the function of the polypeptides encoded by these modified sequences.

The present invention also provides nucleic acids encoding polypeptides and peptides having a modified amino terminus for protection against DPP cleavage and having internal and C-terminus amino acid alterations that do not substantially affect the functional activity or potency of the polypeptide. These modified polypeptides would have minor amino acid changes that are usually conservative amino acid substitutions, although non-conservative substitutions are also contemplated. Nucleotide substitutions using techniques for accomplishing site-specific mutagenesis are well-known in the art. Preferably, the nucleic acids encode GLP-1 analogs having one or more additional amino acids at their N-terminus.

As known in the art “similarity” between two polynucleotides or polypeptides is determined by comparing the nucleotide or amino acid sequence and the conserved nucleotide or amino acid substitutes of one polynucleotide or polypeptide to the sequence of a second polynucleotide or polypeptide. Also known in the art is “identity” which means the degree of sequence relatedness between two polypeptide or two polynucleotide sequences as determined by the identity of the match between two strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

While there exist a number of methods to measure identity and similarity between two polynucleotide or polypeptide sequences, the terms “identity” and “similarity” are well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48: 1073 (1988). Methods commonly employed to determine identity or similarity between two sequences include, but are not limited to those disclosed in Guide to Huge Computers, Martin J. Bishop, ed., Academic Press, San Diego, 1994, and Carillo, H., and Lipman, D., SLAM J. Applied Math. 48:1073 (1988).

Preferred methods to determine identity are designed to give the largest match between the two sequences tested. Methods to determine identity and similarity are codified in computer programs. Preferred computer program methods to determine identity and similarity between two sequences include, but are not limited to, GCG program package (Devereux, et al., Nucleic Acids Research 12(1):387 (1984)), BLASTP, BLASTN, FASTA (Atschul, et al., J. Molec. Biol. 215:403 (1990)). The degree of similarity or identity referred to above is determined as the degree of identity between the two sequences indicating a derivation of the first sequence from the second. The degree of identity between two nucleic acid sequences may be determined by means of computer programs known in the art such as GAP provided in the GCG program package (Needleman and Wunsch (1970) Journal of Molecular Biology 48:443-453). For purposes of determining the degree of identity between two nucleic acid sequences for the present invention, GAP is used with the following settings: GAP creation penalty of 5.0 and GAP extension penalty of 0.3.

Codon Optimization

The degeneracy of the genetic code permits variations of the nucleotide sequence of polypeptides, while still producing a modified polypeptide comprising an identical amino acid sequence as the polypeptide encoded by a first DNA sequence. The procedure, known as “codon optimization” (described in U.S. Pat. No. 5,547,871 which is incorporated herein by reference in its entirety) provides one with a means of designing such an altered DNA sequence. The design of codon optimized genes should take into account a variety of factors, including the frequency of codon usage in an organism, nearest neighbor frequencies, RNA stability, the potential for secondary structure formation, the route of synthesis and the intended future DNA manipulations of that gene. In particular, available methods may be used to alter the codons encoding a given fusion protein with those most readily recognized by yeast when yeast expression systems are used.

The degeneracy of the genetic code permits the same amino acid sequence to be encoded and translated in many different ways. For example, leucine, serine and arginine are each encoded by six different codons, while valine, proline, threonine, alanine and glycine are each encoded by four different codons. However, the frequency of use of such synonymous codons varies from genome to genome among eukaryotes and prokaryotes. For example, synonymous codon-choice patterns among mammals are very similar, while evolutionarily distant organisms such as yeast (S. cerevisiae), bacteria (such as E. coli) and insects (such as D. melanogaster) reveal a clearly different pattern of genomic codon use frequencies (Grantham, R., et al., Nucl. Acids Res., 8, 49-62 (1980); Grantham, R., et al., Nucl. Acids Res., 9, 43-74 (1981); Maroyama, T., et al., Nucl. Acids Res., 14, 151-197 (1986); Aota, S., et al., Nucl. Acids Res., 16, 315-402 (1988); Wada, K., et al., Nucl. Acids Res., 19 Supp., 1981-1985 (1991); Kurland, C. G., FEBS Letters, 285, 165-169 (1991)). These differences in codon-choice patterns appear to contribute to the overall expression levels of individual genes by modulating peptide elongation rates. (Kurland, C. G., FEBS Letters, 285, 165-169 (1991); Pedersen, S., EMBO J., 3, 2895-2898 (1984); Sorensen, M. A., J. Mol. Biol., 207, 365-377 (1989); Randall, L. L., et al., Eur. J. Biochem., 107, 375-379 (1980); Curran, J. F., and Yarus, M., J. Mol. Biol., 209, 65-77 (1989); Varenne, S., et al., J. Mol, Biol., 180, 549-576 (1984), Varenne, S., et al., J. Mol, Biol., 180, 549-576 (1984); Garesl, J.-P., J. Theor. Biol., 43, 211-225 (1974); Ikemura, T., J. Mol. Biol., 146, 1-21 (1981); Ikemura, T., J. Mol. Biol., 151, 389-409 (1981)).

The preferred codon usage frequencies for a synthetic gene should reflect the codon usages of nuclear genes derived from the exact (or as closely related as possible) genome of the cell/organism that is intended to be used for recombinant protein expression, particularly that of yeast species. As discussed above, in one preferred embodiment the modified polypeptide is codon optimized, before or after modification as herein described for yeast expression.

Vectors

Expression units for use in the present invention will generally comprise the following elements, operably linked in a 5′ to 3′ orientation: a transcriptional promoter, a secretory signal sequence, a DNA sequence encoding a modified polypeptide and a transcriptional terminator. As discussed above, any arrangement of the modified polypeptide and peptide may be used in the vectors of the invention. The selection of suitable promoters, signal sequences and terminators will be determined by the selected host cell and will be evident to one skilled in the art and are discussed more specifically below.

Suitable yeast vectors for use in the present invention are described in U.S. Pat. No. 6,291,212 and include YRp7 (Struhl et al., Proc. Natl. Acad. Sci. USA 76: 1035-1039, 1978), YEp13 (Broach et al., Gene 8: 121-133, 1979), pJDB249 and pJDB219 (Beggs, Nature 275:104-108, 1978), pPPC0005, pSeCHSA, pScNHSA, pC4 and derivatives thereof. Useful yeast plasmid vectors also include pRS403-406, pRS413-416 and the Pichia vectors available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, 7RPI, LEU2 and URA3. Plasmids pRS413˜41.6 are Yeast Centromere plasmids (Ycps).

Such vectors will generally include a selectable marker, which may be one of any number of genes that exhibit a dominant phenotype for which a phenotypic assay exists to enable transformants to be selected. Preferred selectable markers are those that complement host cell auxotrophy, provide antibiotic resistance or enable a cell to utilize specific carbon sources, and include LEU2 (Broach et al. ibid.), URA3 (Botstein et al., Gene 8: 17, 1979), HIS3(Struhl et al., ibid.) or POT1 (Kawasaki and Bell, EP 171,142). Other suitable selectable markers include the CAT gene, which confers chloramphenicol resistance on yeast cells. Preferred promoters for use in yeast include promoters from yeast glycolytic genes (Hitzeman et al., J Biol. Chem. 225: 12073-12080, 1980; Alber and Kawasaki, J. Mol. Appl. Genet. 1: 419-434, 1982; Kawasaki, U.S. Pat. No. 4,599,311) or alcohol dehydrogenase genes (Young et al., in Genetic Engineering of Microorganisms for Chemicals, Hollaender et al., (eds.), p. 355, Plenum, N.Y., 1982; Ammerer, Meth. Enzymol. 101: 192-201, 1983). In this regard, particularly preferred promoters are the TPI1 promoter (Kawasaki, U.S. Pat. No. 4,599,311) and the ADH2-4^{c} (see U.S. Pat. No. 6,291,212) promoter (Russell et al., Nature 304: 652-654, 1983). The expression units may also include a transcriptional terminator. A preferred transcriptional terminator is the TPI1 terminator (Alber and Kawasaki, ibid.). More preferably, the promoter is the PRB1 promoter disclosed in EP 431880 and the terminator is the ADH1 terminator disclosed in EP 60057, which are herein incorporated by reference in their entirety.

In addition to yeast, modified polypeptides and peptides of the present invention can be expressed in filamentous fungi, for example, species of the genus Aspergillus. Examples of useful promoters include those derived from Aspergillus nidulans glycolytic genes, such as the ADH3 promoter (McKnight et al., EMBO J. 4: 2093-2099, 1985) and the tpiA promoter. An example of a suitable terminator is the ADH3 terminator (McKnight et al., ibid.). The expression units utilizing such components may be cloned into vectors that are capable of insertion into the chromosomal DNA of Aspergillus, for example.

Mammalian expression vectors for use in carrying out the present invention will include a promoter capable of directing the transcription of the modified polypeptides and peptides. Preferred promoters include viral promoters and cellular promoters. Preferred viral promoters include the major late promoter from adenovirus 2 (Kaufman and Sharp, Mol. Cell. Biol. 2: 1304-13199, 1982) and the SV40 promoter (Subramani et al., Mol. Cell. Biol. 1: 854-864, 1981). Preferred cellular promoters include the mouse metallothionein-1 promoter (Palmiter et al., Science 222: 809-814, 1983) and a mouse Vκ (see U.S. Pat. No. 6,291,212) promoter (Grant et al., Nuc. Acids Res. 15: 5496, 1987). A particularly preferred promoter is a mouse V_{H} (see U.S. Pat. No. 6,291,212) promoter. Such expression vectors may also contain a set of RNA splice sites located downstream from the promoter and upstream from the DNA sequence encoding the modified polypeptide or peptide. Preferred RNA splice sites may be obtained from adenovirus and/or immunoglobulin genes.

Also contained in the expression vectors is a polyadenylation signal located downstream of the coding sequence of interest. Polyadenylation signals include the early or late polyadenylation signals from SV40 (Kaufman and Sharp, ibid.), the polyadenylation signal from the adenovirus 5 E1B region and the human growth hormone gene terminator (DeNoto et al., Nuc. Acids Res. 9: 3719-3730, 1981). A particularly preferred polyadenylation signal is the V_{H} (see U.S. Pat. No. 6,291,212) gene terminator. The expression vectors may include a noncoding viral leader sequence, such as the adenovirus 2 tripartite leader, located between the promoter and the RNA splice sites. Preferred vectors may also include enhancer sequences, such as the SV40 enhancer and the mouse μ (see U.S. Pat. No. 6,291,212) enhancer (Gillies, Cell 33: 717-728, 1983). Expression vectors may also include sequences encoding the adenovirus VA RNAs.

The expression vectors are also used for expressing fusion proteins comprising the modified polypeptide or peptide of the present invention fused to a second polypeptide or peptide, for example transferrin, to enhance the half-life of the modified polypeptide or peptide, as described below. Also, the modified polypeptide or peptide may be fused to a tag and/or a cleavage site for expression and release of the modified polypeptide or peptide.

Transformation

Techniques for transforming fungi are well known in the literature, and have been described, for instance, by Beggs (ibid.), Hinnen et al. (Proc. Natl. Acad. Sci. USA 75: 1929-1933, 1978), Yelton et al., (Proc. Natl. Acad. Sci. USA 81: 1740-1747, 1984), and Russell (Nature 301: 167-169, 1983). The genotype of the host cell will generally contain a genetic defect that is complemented by the selectable marker present on the expression vector. Choice of a particular host and selectable marker is well within the level of ordinary skill in the art.

Cloned DNA sequences comprising modified polypeptides and peptides of the invention may be introduced into cultured mammalian cells by, for example, calcium phosphate-mediated transfection (Wigler et al., Cell 14: 725, 1978; Corsaro and Pearson, Somatic Cell Genetics 7: 603, 1981; Graham and Van der Eb, Virology 52: 456, 1973.) Other techniques for introducing cloned DNA sequences into mammalian cells, such as electroporation (Neumann et al., EMBO J. 1: 841-845, 1982), or lipofection may also be used. In order to identify cells that have integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. A preferred amplifiable selectable marker is the DHFR gene. A particularly preferred amplifiable marker is the DHFR^{r} (see U.S. Pat. No. 6,291,212) cDNA (Simonsen and Levinson, Proc. Natl. Acad. Sci. USA 80: 2495-2499, 1983). Selectable markers are reviewed by Thilly (Mammalian Cell Technology, Butterworth Publishers, Stoneham, Mass.) and the choice of selectable markers is well within the level of ordinary skill in the art.

Host Cells

The present invention also includes a cell, preferably a yeast cell transformed to express a modified polypeptides or peptides of the invention. In addition to the transformed host cells themselves, the present invention also includes a culture of those cells, preferably a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away.

Host cells for use in practicing the present invention include eukaryotic cells, and in some cases prokaryotic cells, capable of being transformed or transfected with exogenous DNA and grown in culture, such as cultured mammalian, insect, fungal, plant and bacterial cells. A vector comprising a nucleic acid sequence of the present invention is introduced into a host cell so that the vector is maintained as a chromosomal integrant or as a self-replicating extra-chromosomal vector. Integration is generally considered to be an advantage as the nucleic acid sequence is more likely to be stably maintained in the cell. Integration of the vector into the host chromosome may occur by homologous or non-homologous recombination.

The choice of a host cell will to a large extent depend upon the gene encoding the polypeptide and its source. The host cell may be a unicellular microorganism, e.g., a prokaryote, or a non-unicellular microorganism, e.g., a eukaryote. Either prokaryotes or eukaryotes can be used. As prokaryotic host cells, generally used cells such as Escherichia coli or Bacillus subtilis can be used.

When prokaryotic cells are used as host cells, a vector replicable in the host cells may be used. An expression plasmid can be preferably used in which a promoter, an SD sequence (Shine-Delgarno sequence), and an initiation codon (e.g. ATG) required for starting protein synthesis are provided in the vector upstream of the gene of the present invention to facilitate expression of the gene. Examples of the above vector include generally-used plasmids derived from E. coli such as pBR322, pBR325, pUC12, pUC13 and the like. However, applicable vectors are not limited to these examples and various known vectors can also be used. Examples of commercially available vectors usable in expression system using E. coli include pGEX-4T (Amersham Pharmacia Biotech), pMAL-C2, pMA1-P2 (New England Biolabs), pET21/lacq (Invitrogen), pBAD/His (Invitrogen) and the like.

Examples of eukaryotic host cells include yeast cells and the like. Examples of preferably used craniate cells include COS cell (cell from monkey) (1981 Cell, 23, 175), Chinese Hamster Ovary cells and the dihydrofolate reductase defective strain derived therefrom (1980 Proc. Natl. Acad. Sci., USA., 77, 4216) and the like, and examples of preferably used yeast cells include Saccharomyces cerevisiae or the like. However, cells to be used are not limited to these examples. Preferably, a yeast cell is used to express the modified polypeptide or peptide.

Fungal cells, including species of yeast (e.g., Saccharomyces spp., Schizosaccharomyces spp., Pichia spp.) may be used as host cells within the present invention. Examples of fungi including yeasts contemplated to be useful in the practice, of the present invention as hosts for expressing the modified polypeptide or peptides of the inventions are Pichia (including species formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschnikowia, Rhodosporidium, Leucosporidium, Botryoascus, Sporidiobolus, Endomycopsis, and the like. Examples of Saccharomyces spp. are S. cerevisiae, S. italicus and S. rouxii. Examples of KIuyveromyces spp. are K. fragilis, K. lactis and K. marxianus. A suitable Torulaspora species is T. delbrueckii. Examples of Pichia spp. are P. angusta (formerly H. polymorpha), P. anomala (formerly H. anomala) and P. pastoris.

Particularly useful host cells to produce the modified polypeptide or peptide of the invention are the methanoltrophic Pichia pastoris (Steinlein et al. (1995) Protein Express. Purif. 6:619-624). Pichia pastoris has been developed to be an outstanding host for the production of foreign proteins since its alcohol oxidase promoter was isolated and cloned; its transformation was first reported in 1985. P. pastoris can utilize methanol as a carbon source in the absence of glucose. The P. pastoris expression system can use the methanol-induced alcohol oxidase (AOX1) promoter, which controls the gene that codes for the expression of alcohol oxidase, the enzyme which catalyzes the first step in the metabolism of methanol. This promoter has been characterized and incorporated into a series of P. pastoris expression vectors. Since the proteins produced in P. pastoris are typically folded correctly and secreted into the medium, the fermentation of genetically engineered P. pastoris provides an excellent alternative to E. coli expression systems.

Strains of the yeast Saccharomyces cerevisiae are another preferred host. In a preferred embodiment, a yeast cell, or more specifically, a Saccharomyces cerevisiae host cell that contains a genetic deficiency in a gene required for asparagine-linked glycosylation of glycoproteins is used. S. cerevisiae host cells having such defects may be prepared using standard techniques of mutation and selection, although many available yeast strains have been modified to prevent or reduce glycosylation or hypermannosylation.

To optimize production of the heterologous proteins, it is also preferred that the host strain carry a mutation, such as the S. cerevisiae pep4 mutation (Jones, Genetics 85: 23-33, 1977), which results in reduced proteolytic activity. It is particularly advantageous to use a host that carries a mutation in the gene encoding the aspartyl protease yapsin 1(YAP3) or the gene encoding yapsin 2(MKC7), or both (Copley et al. 1998 Biochem. J. 330, 1333-1340), such that the proteolytic activity directed to basic residues is reduced or eliminated. Host strains containing mutations in other protease encoding regions are particularly useful to produce large quantities of the modified therapeutic polypeptides or peptides of the invention.

Host cells containing DNA constructs of the present invention are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals and growth factors. The growth medium will generally select for cells containing the DNA construct by, for example, drug selection or deficiency in an essential nutrient which is complemented by the selectable marker on the DNA construct or co-transfected with the DNA construct. Yeast cells, for example, are preferably grown in a chemically defined medium, comprising a non-amino acid nitrogen source, inorganic salts, vitamins and essential amino acid supplements. The pH of the medium is preferably maintained at a pH greater than 2 and less than 8, preferably at pH 5.5 to 6.5. Methods for maintaining a stable pH include buffering and constant pH control, preferably through the addition of ammonia, ammonium hydroxide or sodium hydroxide. Preferred buffering agents include citric acid, phosphate, succinic acid and Bis-Tris (Sigma Chemical Co., St. Louis, Mo.). Yeast cells having a defect in a gene required for asparagine-linked glycosylation are preferably grown in a medium containing an osmotic stabilizer. A preferred osmotic stabilizer is sorbitol supplemented into the medium at a concentration between 0.1 M and 1.5 M., preferably at 0.5 M or 1.0 M.

Cultured mammalian cells are generally grown in commercially available serum-containing or serum-free medium. Selection of a medium appropriate for the particular cell line used is within the level of ordinary skill in the art. Transfected mammalian cells are allowed to grow for a period of time, typically 1-2 days, to begin expressing the DNA sequence(s) of interest. Drug selection is then applied to select for growth of cells that are expressing the selectable marker in a stable fashion. For cells that have been transfected with an amplifiable selectable marker the drug concentration may be increased in a stepwise manner to select for increased copy number of the cloned sequences, thereby increasing expression levels.

Baculovirus/insect cell expression systems may also be used to produce the modified therapeutic polypeptides or peptides of the invention. The BacPAK™ Baculovirus Expression System (BD Biosciences (Clontech) expresses recombinant proteins at high levels in insect host cells. The target gene is inserted into a transfer vector, which is cotransfected into insect host cells with the linearized BacPAK6 viral DNA. The BacPAK6 DNA is missing an essential portion of the baculovirus genome. When the DNA recombines with the vector, the essential element is restored and the target gene is transferred to the baculovirus genome. Following recombination, a few viral plaques are picked and purified, and the recombinant phenotype is verified. The newly isolated recombinant virus can then be amplified and used to infect insect cell cultures to produce large amounts of the desired protein.

Secretory Signal Sequences

The terms “secretory signal sequence” or “signal sequence” or “secretion leader sequence” are used interchangeably and are described, for example in U.S. Pat. Nos. 6,291,212 and 5,547,871, both of which are herein incorporated by reference in their entirety. Secretory signal sequences or signal sequences or secretion leader sequences encode secretory peptides. A secretory peptide is an amino acid sequence that acts to direct the secretion of a mature polypeptide or protein from a cell. Secretory peptides are generally characterized by a core of hydrophobic amino acids and are typically (but not exclusively) found at the amino termini of newly synthesized proteins. Very often the secretory peptide is cleaved from the mature protein during secretion. Secretory peptides may contain processing sites that allow cleavage of the signal peptide from the mature protein as it passes through the secretory pathway. Processing sites may be encoded within the signal peptide or may be added to the signal peptide by, for example, in vitro mutagenesis.

Secretory peptides may be used to direct the secretion of modified polypeptides and peptides of the invention. One such secretory peptide that may be used in combination with other secretory peptides is the third domain of the yeast Barrier protein. Secretory signal sequences or signal sequences or secretion leader sequences are required for a complex series of post-translational processing steps which result in secretion of a protein. If an intact signal sequence is present, the protein being expressed enters the lumen of the rough endoplasmic reticulum and is then transported through the Golgi apparatus to secretory vesicles and is finally transported out of the cell. Generally, the signal sequence immediately follows the initiation codon and encodes a signal peptide at the amino-terminal end of the protein to be secreted. In most cases, the signal sequence is cleaved off by a specific protease, called a signal peptidase. Preferred signal sequences improve the processing and export efficiency of recombinant protein expression using viral, mammalian or yeast expression vectors. A preferred signal sequence is a mammalian or human transferrin signal sequence. In some cases, the native substrate signal sequence may be used to express and secrete modified polypeptide or peptides of the invention. In order to ensure efficient removal of the signal sequence, in some cases it may be preferable to include a short pro-peptide sequence between the signal sequence and the mature protein in which the C-terminal portion of the pro-peptide comprises a recognition site for a protease, such as the yeast kex2p protease. Preferably, the pro-peptide sequence is about 2-12 amino acids in length, more preferably about 4-8 amino acids in length. Examples of such pro-peptides are Arg-Ser-Leu-Asp-Lys-Arg, Arg-Ser-Leu-Asp-Arg-Arg, Arg-Ser-Leu-Glu-Lys-Arg, and Arg-Ser-Leu-Glu-Arg-Arg (SEQ ID NOS: 111-114, respectively).

Production of Modified Polypeptide Substrates Protected from DPP Cleavage

The modified polypeptides of this invention that are partially or substantially resistant to DPP activity, may be prepared by standard synthetic methods, recombinant DNA techniques, or any other methods of preparing peptides and fusion proteins.

The solid phase peptide synthesis method is generally described in the following references: Merrifield, J. Am. Chem. Soc., 888:2149, 1963; Barany and Merrifield, In the Peptides, E. Gross and J. Meinenhofer, Eds., Academic Press, New York, 3:285 (1980); S. B. H. Kent. Annu. Rev. Biochem., 57:957 (1988). By the solid phase peptide synthesis method, a peptide of a desired length and sequence can be produced through the stepwise addition of amino acids to a growing peptide chain which is covalently bound to a solid resin particle. Automated synthesis may be employed in this method.

As discussed above, the modified polypeptide of the present invention may also be obtained using molecular biology techniques, employing nucleic acid sequences that encode those polypeptides. Those sequences may be RNA or DNA and may be associated with control sequences and/or inserted into vectors. The latter are then transfected into host cells, for example bacteria. The preparation of the vectors and their production or expression in a host is carried out by conventional molecular biology and genetic engineering techniques.

Moreover, the modified polypeptides of the present invention can also be made by recombinant techniques using readily synthesized DNA sequences in commercially available expression systems.

The modified polypeptides of the present invention may be obtained by recombinant means comprising (a) cultivating a host cell under conditions conducive to production of the polypeptide; and (b) recovering the polypeptide. The cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art (see, e.g., references for bacteria and yeast; Bennett, J. W. and LaSure, L., editors, More Gene Manipulations in Fungi, Academic Press, California, 1991). Suitable media are available from commercial suppliers or may be prepared according to published compositions (e.g., in catalogues of the American Type Culture Collection). If the modified polypeptide is secreted into the nutrient medium, the polypeptide can be recovered directly from the medium. If the modified polypeptide is not secreted, it can be recovered from cell lysates.

As an example, the modified polypeptides or peptides of the present invention including the modified polypeptide or peptide fusion protein may be made by the fermentation methodology disclosed in WO 0044772, which is herein incorporated by reference in its entirety.

The modified polypeptides may be detected using methods known in the art that are specific for the polypeptides. These detection methods may include use of specific antibodies, formation of an enzyme product, or disappearance of an enzyme substrate, binding to a specific receptor, or by detection of activation of a specific receptor in a cell-based assay. For example, an enzyme assay may be used to determine the activity of the modified polypeptide. The resulting modified polypeptide may be recovered by methods known in the art. For example, the modified polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing, differential solubility (e.g., ammonium sulfate precipitation), SDS-PAGE, or extraction (see, e.g., Protein Purification, J.-C. Janson and Lars Ryden, editors, VCH Publishers, New York, 1989).

Fusion Proteins and Protein Conjugates.

The present invention provides modified polypeptides or peptides attached to a heterologous molecule via recombinant means or covalent attachment. The attachment to a heterologous molecule, for example a plasma protein, extends the activity of the modified polypeptides or peptides for days to weeks. In some instances, only one administration of such modified therapeutic polypeptide or peptide need be given during this period of time. Greater specificity can be achieved, since the active compound will be primarily bound to large molecules, where it is less likely to be taken up intracellularly to interfere with other physiological processes.

In another embodiment, the modified polypeptides or peptides of the present invention can be attached to heterologous sequences to form chimeric or fusion proteins via recombinant means. Such chimeric or fusion proteins comprise a modified polypeptide or peptide, partially or substantially protected from DPP cleavage, operatively linked to a heterologous protein having an amino acid sequence not substantially homologous to the modified polypeptide or peptide. “Operatively linked” indicates that the modified polypeptide or peptide and the heterologous protein are fused in-frame. The heterologous protein can be fused to the N-terminus or C-terminus of the modified polypeptide or peptide.

In one embodiment, the fusion protein does not affect the activity of the modified polypeptide of the invention per se. For example, the fusion protein can include, but is not limited to, enzymatic fusion proteins, for example beta-galactosidase fusions, yeast two-hybrid GAL fusions, poly-His fusions, MYC-tagged, HI-tagged and Ig fusions. Such fusion proteins, particularly poly-His fusions, can facilitate the purification of recombinant modified polypeptide. In a further example, the fusion protein comprises an amino acid sequence between the modified peptide of the invention and the other moiety, said amino acid sequence providing a recognition sequence that enables release of the modified peptide of the invention following chemical or enzymatic cleavage. In certain host cells (e.g., mammalian host cells), expression and/or secretion of a protein can be increased by using a heterologous signal sequence. In another embodiment, the modified polypeptide or peptide is fused to a molecule that will extend its serum stability or serum half-life, such as a plasma protein. Preferably, the modified polypeptide or protein is fused to serum albumin, immunoglobulin, or a portion thereof such as the Fc domain. More preferably, the modified polypeptide or peptide is fused to transferrin, lactotransferrin, melanotransferrin, or hybrids thereof. Methods for making such fusion proteins are provided by U.S. applications Ser. No. 10/231,494 and Ser. No. 10/378,094, and International Application PCT/US03/26818, which are herein incorporated by reference in their entirety.

As discussed in these applications, the transferrin to be attached to the modified polypeptide or peptide may be modified. It may exhibit reduced glycosylation. The modified transferrin polypeptide may be selected from the group consisting of a single transferrin N domain, a single transferrin C domain, a transferrin N and C doman, two transferrin N domains, and two transferrin C domains.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al. 1992 Current Protocols in Molecular Biology). Moreover, many expression vectors are commercially available that already encode a fusion moiety (e.g., a GST protein). A modified polypeptide or peptide encoding nucleic acid can be cloned into such an expression vector such that the fusion moiety is linked in-frame to the modified polypeptide or peptide.

In another embodiment, the modified therapeutic polypeptide or peptide is conjugated via a covalent bond to a heterologous molecule via a covalent bond to increase its stability and protection from DPP activity.

As an example, the modified polypeptide or peptide is conjugated to a blood component via a covalent bond formed between the reactive group of the modified peptide and a blood component, with or without a linking group. Blood components may be either fixed or mobile. Examples of fixed blood components are non-mobile blood components and include tissues, membrane receptors, interstitial proteins, fibrin proteins, collagens, platelets, endothelial cells, epithelial cells and their associated membrane and membraneous receptors, somatic body cells, skeletal and smooth muscle cells, neuronal components, osteocytes and osteoclasts and all body tissues especially those associated with the circulatory and lymphatic systems. Example of mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, immunoglobulins such as IgM and IgG, α₁ protease inhibitor, antithrombin III and α₂-antiplasmin. The half-life of mobile blood components is typically at least about 12 hours.

The formation of the covalent bond between the blood component and the modified therapeutic polypeptide or peptide may occur in vivo or ex vivo. For ex vivo covalent bond formation, the modified polypeptide or peptide is added to blood, serum or saline solution containing the blood component, e.g. human serum albumin or IgG to permit covalent bond formation between the modified polypeptide or peptide and the blood component. Also, the modified polypeptide peptide may be modified with maleimide or a similarly reactive chemical group and reacted with a blood component in saline solution. Once the modified therapeutic polypeptide or peptide is reacted with the blood component to form a modified polypeptide or peptide conjugate, the conjugate may be administered to the patient. Alternatively, the modified therapeutic polypeptide or peptide may be administered to the patient directly so that the covalent bond forms between the modified therapeutic polypeptide or peptide and the blood component in vivo. Also, the same reaction may be carried out with a recombinant protein, for example, albumin.

The various sites with which the chemically reactive groups of the non-specific modified therapeutic polypeptide or peptide may react in vivo include cells, particularly red blood cells (erythrocytes) and platelets, and proteins, such as immunoglobulins, including IgG and IgM, serum albumin, ferritin, steroid binding proteins, transferrin, thyroxin binding protein, α-2-macroglobulin, and the like.

The modified polypeptide or peptide may contain or may be chemically modified to contain a reactive group for binding to thiol. In one embodiment of the invention the modified polypeptide or peptide may be conjugated to polyethylene glycol. Alternatively, the modified polypeptide or peptide may be conjugated to a polyethylene glycol modified glycolipid or polyethylene glycol modified fatty acid.

In one aspect, the modified polypeptide or peptide may be conjugated to a fatty acid or fatty acid derivative to improve its stability. Examples of fatty acids include, but are not limited to, lauric, palmitic, oleic, and stearic acids. Examples of fatty acid derivatives include ethyl esters, propyl esters, cholesteryl esters, coenzyme A esters, nitrophenyl esters, naphthyl esters, monoglycerides, diglycerides, and triglycerides, fatty alcohols, fatty alcohol acetates, and the like.

In another aspect, the modified polypeptide or peptide may be engineered into into a drug affinity complex (DAC™). A drug affinity complex has three parts: a drug component which is responsible for biological activity; a connector attaching the drug component to the reactive chemistry group; and a reactive chemistry group, at the the opposite end of the connector, which is responsible for the permanent bonding of the construct to certain target proteins in the body. For example, Kim et al. (2003, Diabetes 52(3):751) disclose a GLP-1-albumin drug affinity complex. Kim et al. show that the albumin-conjugated DAC:GLP-1 bound to the GLP-1 receptor (GLP-1R) and activated cAMP formation in heterologous fibroblasts expressing the receptor. The results suggest that the albumin-conjugated DAC:GLP-1 mimics the native GLP-1. Kim et al. provide a new approach for prolonged activation of GLP-1R signaling.

The modified polypeptide or peptide drug affinity complex is designed to be administered by subcutaneous injection and then rapidly and selectively bonds in vivo to albumin. The bioconjugate formed has the same therapeutic activity and similar potency as endogenous polypeptide or peptide but has a pharmacokinetic profile in animals that is closer to that of albumin.

Pharmaceutical Composition

The present invention provides pharmaceutical compositions comprising modified therapeutic polypeptides and peptides partially or substantially protected from DPP cleavage, but substantially retaining their functional activity and potency. Such pharmaceutical compositions may be be administered orally, parenterally, such as intravascularly (IV), intraarterially (IA), intramuscularly (IM), subcutaneously (SC), intraperitoneally, transdermally, or the like. Administration may in appropriate situations be by transfusion. In some instances, administration may be oral, nasal, rectal, transdermal or aerosol, where the modified polypeptide allows for transfer to the vascular system. For example, fusion or conjugation of a modified polypeptide of the invention to a transferrin moiety allows for transport of the modified polypeptide to the vascular system or across the blood-brain barrier via binding to the transferrin receptor, as described in International Application PCT/US03/26778, which is herein incorporated by reference in its entirety. Usually a single injection will be employed although more than one injection may be used, if desired. The modified therapeutic polypeptides or peptides may be administered by any convenient means, including syringe, trocar, catheter, or the like. The particular manner of administration will vary depending upon the amount to be administered, whether a single bolus or continuous administration, or the like. Preferably, the administration will be intravascularly, where the site of introduction is not critical to this invention, preferably at a site where there is rapid blood flow, e.g., intravenously, peripheral or central vein. More preferably, the pharmaceutical compositions will be administered subcutaneously. Other routes may find use where the administration is coupled with slow release techniques or a protective matrix. The intent is that the modified therapeutic peptides or polypeptides be effectively distributed, for example, in the blood, so as to be able to react with the blood or tissue components.

Generally, the invention encompasses pharmaceutical compositions comprising effective amounts of modified therapeutic polypeptide or peptide of the invention together with pharmaceutically acceptable diluents, preservatives, solubilizers, emulsifiers, adjuvants and/or carriers. Such compositions may include diluents of various buffer content (e.g., Tris-HCl, acetate, phosphate), pH and ionic strength; additives such as detergents and solubilizing agents (e.g., Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference.

For example, the modified therapeutic polypeptides or peptides may be administered in a physiologically acceptable medium, e.g., deionized water, phosphate buffered saline (PBS), saline, aqueous ethanol or other alcohol, plasma, proteinaceous solutions, mannitol, aqueous glucose, alcohol, vegetable oil, or the like. Other additives which may be included include buffers, where the media are generally buffered at a pH in the range of about 5 to 10, where the buffer will generally range in concentration from about 50 to 250 mM, salt, where the concentration of salt will generally range from about 5 to 500 mM, physiologically acceptable stabilizers, and the like. Examples of physiological buffers, especially for injection, include Hank's solution and Ringer's solution. Transdermal formulations may contain penetrants such as bile salts or fusidates.

The pharmaceutical compositions may be prepared as tablets or dragees, sublingual tablets, sachets, paquets, soft gelatin capsules, suppositories, creams, ointments, dermal gels, transdermal devices, aerosols, drinkable and injectable ampoules. The compositions may also be prepared in liquid form, or may be in dried powder, such as lyophilized form convenient for storage and transport. Implantable sustained release formulations are also contemplated.

Oral Dosage Forms

In one embodiment, the present invention provides pharmaceutical compositions comprising the modified therapeutic polypeptides or peptides in oral solid dosage forms, which are described generally in Remington's Pharmaceutical Sciences (1990), 18th Ed., Mack Publishing Co. Easton Pa. 18042, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given in Chapter 10 of Marshall, K., Modern Pharmaceutics (1979), edited by G. S. Banker and C. T. Rhodes, herein incorporated by reference. In general, the formulation will include the modified therapeutic polypeptide or peptide, and inert ingredients which allow for protection against the stomach environment, and release of the biologically active material in the intestine.

If necessary, the modified therapeutic polypeptide or peptide may be chemically modified so that oral delivery is efficacious. Generally, the chemical modification contemplated is the attachment of at least one moiety to the modified therapeutic polypeptide or peptide itself, where said moiety permits uptake into the blood stream from the stomach or intestine. Also desired is the increase in overall stability of the compound and increase in circulation time in the body. Moieties useful as covalently attached vehicles in this invention may also be used for this purpose. Examples of such moieties include: PEG, copolymers of ethylene glycol and propylene glycol, carboxymethyl cellulose, dextran, polyvinyl alcohol, polyvinyl pyrrolidone and polyproline. See, for example, Abuchowski and Davis, Soluble Polymer-Enzyme Adducts, Enzymes as Drugs (1981), Hocenberg and Roberts, eds., Wiley-Interscience, New York, N.Y., pp 367-83; Newmark, et al. (1982), J. Appl. Biochem. 4:185-9. Other polymers that could be used are poly-1,3-dioxolane and poly-1,3,6-tioxocane. Preferred for pharmaceutical usage, as indicated above, are PEG moieties.

Likewise, the modified therapeutic polypeptide or peptide may be recombinantly fused to another polypeptide to increase its overall stability or improve oral delivery. For example, the modified therapeutic polypeptide or peptide may be fused to transferrin, melanotransferrin, or lactoferrin. Methods for making such fusion proteins are described in U.S. application Ser. No. 10/378,094, which is herein incorporated by reference in its entirety.

For oral delivery dosage forms, it is also possible to use a salt of a modified aliphatic amino acid, such as sodium N-(8-[2-hydroxybenzoyl] amino) caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods” which is herein incorporated by reference in its entirety.

The modified therapeutic polypeptides or peptides of this invention can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the modified therapeutic polypeptide or peptide may be formulated (such as by liposome or microsphere encapsulation) and then further contained within an edible product, such as a refrigerated beverage containing colorants and flavoring agents.

One may dilute or increase the volume of the pharmaceutical composition of the invention with an inert material. These diluents could include carbohydrates, especially mannitol, cc-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the modified therapeutic polypeptide or peptide together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the pharmaceutical composition of the invention to prevent sticking during the formulation process. Lubricants may be used as a layer between the modified therapeutic polypeptide or peptide and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the modified therapeutic polypeptide or peptide during formulation and to aid rearrangement during compression might be added. The glidants may include starch, talc, pyrogenic silica and hydrated silicoaluminate.

To aid dissolution of the modified therapeutic polypeptide or peptide of this invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives may also be included in the formulation to enhance uptake of the modified therapeutic polypeptide and peptide. Additives potentially having this property are for instance the fatty acids oleic acid, linoleic acid and linolenic acid.

Controlled release formulation also may be desirable. The modified therapeutic polypeptide or peptide of this invention could be incorporated into an inert matrix which permits release by either diffusion or leaching mechanisms e.g., gums. Slowly degenerating matrices may also be incorporated into the formulation, e.g., alginates, polysaccharides. Another form of a controlled release of the compounds of this invention is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The modified therapeutic polypeptide or peptide could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Pulmonary Delivery Forms

In another embodiment, the present invention also provides pharmaceutical compositions comprising the modified therapeutic polypeptides or peptides for pulmonary delivery. The pharmaceutical composition is delivered to the lungs of a mammal while inhaling and traverses across the lung epithelial lining to the blood stream.

The present invention provides the use of a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the modified therapeutic polypeptide and peptide. Typically, each formulation is specific to the type of device employed and may involve the use of an appropriate propellant material, in addition to diluents, adjuvants and/or carriers useful in therapy.

The modified therapeutic polypeptide or peptide should most advantageously be prepared in particulate form with an average particle size of less than 10 μm, most preferably 0.5 to 5 μm, for most effective delivery to the distal lung.

Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise the inventive compound dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the inventive compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent, such as lactose, sorbitol, sucrose, mannitol, trehalose, or xylitol in amounts which facilitate dispersal of the powder from the device, e.g., about 50 to 90% by weight of the formulation.

Nasal Delivery Forms

Nasal delivery of the pharmaceutical composition of the modified polypeptide or peptide of the present invention is also contemplated. Nasal delivery allows the passage of the protein to the blood stream directly after administering the modified therapeutic polypeptide or peptide to the nose, without the necessity for deposition of the product in the lung. Formulations for nasal delivery include those with dextran or cyclodextran. Delivery via transport across other mucous membranes is also disclosed.

Dosages

The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.01-1000 micrograms of the inventive compound per kilogram of body weight, preferably 0.1-150 micrograms per kilogram.

Treatment of Diseases with Modified Therapeutic Proteins

The present invention provides various modified therapeutic polypeptides or peptides that could be used in the treatment of a variety of diseases. For example, the pharmaceutical compositions comprising the modified therapeutic polypeptides or peptides of the present invention could be used to treat diseases such as, but not limited, to insulin resistance, hyperglycemia, hyperinsulinemia, or elevated blood levels of free fatty acids or glycerol, hyperlipidemia, obesity, Syndrome X, dysmetabolic syndrome, inflammation, diabetic complications, impaired glucose homeostasis, impaired glucose tolerance, hypertriglyceridemia atherosclerosis, nervous system disorders. The modified polypeptides and peptides could also be used to induce an anxiolytic effect on the CNS, to activate the CNS or for post surgery treatment.

The modified therapeutic polypeptides and peptides of the present invention are more stable in vivo than the nonmodified therapeutic polypeptides and peptides because they are partially or substantially protected from DPP activity. Accordingly, smaller amounts of the molecule may be administered for effective treatment. A lower dosage amount may in some instances alleviate side effects.

In one embodiment, the modified therapeutic polypeptides and peptides of the present invention may be used as a sedative. Accordingly, the present invention provides a method of sedating a mammalian subject with an abnormality resulting in increased activation of the central or peripheral nervous system using the modified polypeptides or peptides of the invention. The method comprises administering a modified therapeutic polypeptides or peptides to the subject in an amount sufficient to produce a sedative or anxiolytic effect on the subject. The modified therapeutic polypeptides or peptides may be administered intracerebroventriculary, orally, subcutaneously, intramuscularly, or intravenously. Such methods are useful to treat or ameliorate nervous system conditions such as anxiety, movement disorder, aggression, psychosis, seizures, panic attacks, hysteria and sleep disorders.

Moreover, the present invention encompasses a method of increasing the activity of a mammalian subject, comprising administering a modified therapeutic polypeptides or peptides to the subject in an amount sufficient to produce an activating effect on the subject. The subject has a condition resulting in decreased activation of the central or peripheral nervous system. The modified therapeutic polypeptides or peptides are useful in the treatment or amelioration of depression, schizoaffective disorders, sleep apnea, attention deficit syndromes with poor concentration, memory loss, forgetfulness, and narcolepsy, to name just a few conditions in which arousal of the central nervous system may be advantageous.

Also, insulin resistance following a particular type of surgery, elective abdominal surgery, is most profound on the first post-operative day, lasts at least five days, and may take up to three weeks to normalize. Thus, the post-operative patient may be in need of administration of the modified insulinotropic peptides of the present invention for a period of time following the trauma of surgery. Accordingly, the modified therapeutic polypeptides or peptides of the invention may be utilized for post surgery treatments. A patient is in need of the modified insulinotropic peptides of the present invention for about 1-16 hours before surgery is performed on the patient, during surgery on the patient, and after the patient's surgery for a period of not more than about 5 days.

Moreover, the modified therapeutic polypeptides and peptides, such as the insulinotropic peptides, of the invention may be utilized to treat insulin resistance independently from their use in post surgery treatment. Insulin resistance may be due to a decrease in binding of insulin to cell-surface receptors, or to alterations in intracellular metabolism. The first type, characterized as a decrease in insulin sensitivity, can typically be overcome by increased insulin concentration. The second type, characterized as a decrease in insulin responsiveness, cannot be overcome by large quantities of insulin. Insulin resistance following trauma can be overcome by doses of insulin that are proportional to the degree of insulin resistance, and thus is apparently caused by a decrease in insulin sensitivity.

Preferably, the present invention provides modified insulinotropic peptides to normalize hyperglycemia through glucose-dependent, insulin-dependent and insulin-independent mechanisms. As such, the modified insulinotropic peptides are useful as primary agents for the treatment of diabetes, especially type II diabetes mellitus. The present invention is especially suited for the treatment of patients with diabetes, both type I and type II, in that the action of the peptide is dependent on the glucose concentration of the blood, and thus the risk of hypoglycemic side effects are greatly reduced over the risks in using current methods of treatment

The dose of modified insulinotropic peptides effective to normalize a patient's blood glucose level will depend on a number of factors, among which are included, without limitation, the patient's sex, weight and age, the severity of inability to regulate blood glucose, the underlying causes of inability to regulate blood glucose, whether glucose, or another carbohydrate source, is simultaneously administered, the route of administration and bioavailability, the persistence in the body, the formulation, and the potency.

Preferably, the modified therapeutic peptides such as the insulinotropic peptides, of the present invention are used for the treatment of impaired glucose tolerance, glycosuria, hyperlipidaemia, metabolic acidoses, diabetes mellitus, diabetic neuropathy, and nephropathy. More preferably, the modified peptides are modified GLP-1 and analogs thereof for the treatment of type II diabetes.

Monitoring the Presence of Modified Therapeutic Polypeptides and Peptides

The modified therapeutic polypeptides and peptides may be monitored using assays for determining functional activity, HPLC-MS, or antibodies directed against the polypeptide or peptide. For example, the blood of the mammalian host may be monitored for the activity of the modified therapeutic polypeptide or peptide and/or presence of the modified therapeutic polypeptide or peptide. By taking a portion or sample of the blood of the host at different times, one may determine whether the modified therapeutic polypeptide or peptide has become bound to the long-lived blood components in sufficient amount to be therapeutically active and, thereafter, the level of modified therapeutic polypeptide or peptide in the blood. If desired, one may also determine to which of the blood components the modified therapeutic polypeptide or peptide, such as a modified insulinotropic peptide, is bound.

As an example, assays for insulinotropic activity may be used to monitor the modified insulinotropic peptides of the present invention. The modified insulinotropic peptides of the present invention have an insulinotropic activity that at least equals the insulinotropic activity of the non-modified insulinotropic peptides. The insulinotropic property of a modified insulinotropic peptide may be determined by providing that modified peptide to animal cells, or injecting that peptide into animals and monitoring the release of immunoreactive insulin into the media or circulatory system of the animal, respectively. The presence of immunoreactive insulin is detected through the use of a radioimmunoassay which can specifically detect insulin. Although any radioimmunoassay capable of detecting the presence of IRI may be employed, it is preferable to use a modification of the assay method of Albano, J. D. M., et al., (1972 Acta Endocrinol. 70:487-509), which is herein incorporated by reference in its entirety.

The insulinotropic property of a modified therapeutic polypeptide or peptide may also be determined by pancreatic infusion (Penhos, J. C., et al. 1969 Diabetes 18:733-738, which is hereby incorporated by reference). The manner in which perfusion is performed, modified, and analyzed preferably follows the methods of Weir, G. C., et al., (J. Clin. Investigat. 54:1403-1412 (1974)), which is hereby incorporated by reference.

HPLC coupled with mass spectrometry (MS) can be utilized to assay for the presence of modified therapeutic polypeptide and peptides as is well known to the skilled artisan. Typically two mobile phases are utilized, such as 0.1% TFA/water and 0.1% TFA/acetonitrile. Column temperatures can be varied as well as gradient conditions.

Another method to monitor the presence of modified therapeutic polypeptides and peptides is to use antibodies specific to the modified therapeutic polypeptides and peptides. The use of antibodies, either monoclonal or polyclonal, having specificity for particular modified therapeutic polypeptides or peptides, can assist in mediating any such problem. The antibody may be generated or derived from a host immunized with the particular modified therapeutic polypeptide or peptide, or with an immunogenic fragment of the agent, or a synthesized immunogen corresponding to an antigenic determinant of the agent. Preferred antibodies will have high specificity and affinity for the modified therapeutic polypeptide or peptide. Such antibodies can also be labeled with enzymes, fluorochromes, or radiolabels.

The antibodies may be used to monitor the presence of modified therapeutic polypeptides and peptides in the blood stream. Blood and/or serum samples may be analyzed by SDS-PAGE and western blotting. Such techniques permit the analysis of the blood or serum to determine the bonding of the modified therapeutic polypeptides or peptides to blood components.

Glucagon-Like Peptide-1 (GLP-1)

Preferably, the modified therapeutic peptides of the present invention are modified insulinotropic peptides partially or substantially protected from DPP activity. More preferably, the modified insulinotropic peptides are modified GLP-1 peptides and analogs and fragments thereof. The modified GLP-1 peptides and analogs and fragments thereof are useful for treating diabetes, specifically type II diabetes. The N-terminal sequence of wild-type GLP-1 is His-Ala-Glu; preferred modified GLP-1 polypeptides of the invention comprise an N-terminal sequence selected from the group consisting of: His-His-Ala-Glu (SEQ ID NO: 115), Gly-His-Ala-Glu (SEQ ID NO: 116), His-Gly-Glu, His-Ser-Glu, His-Ala-Glu, His-Gly-Glu, His-Ser-Glu, His-His-Ala-Glu (SEQ ID NO: 82), His-His-Gly-Glu (SEQ ID NO: 83), His-His-Ser-Glu (SEQ ID NO: 84), Gly-His-Ala-Glu (SEQ ID NO: 85), Gly-His-Gly-Glu (SEQ ID NO: 86), Gly-His-Ser-Glu (SEQ ID NO: 87), His-X-Ala-Glu, His-X-Gly-Glu, and His-X-Ser-Glu, wherein X is any amino acid.

The C-terminus of GLP-1 is normally amidated. In yeast, amidation does not occur. In one aspect of the invention, in order to compensate for amidation on the N-terminus which does not occur in yeast, an extra amino acid is added on the N-terminus of GLP-1. The addition of an amino acid to the N-terminus of GLP-1 may prevent dipeptidyl peptidase from cleaving at the second amino acid of GLP-1 due to steric hindrance. Therefore, GLP-1 will remain functionally active. Any one of the 20 amino acids or a non-natural amino acid may be added to the N-terminus of GLP-1. Histidine is also a preferred amino acid. In some instances, an uncharged or positively charged amino acid may be used and preferably, a smaller amino acid such as Glycine is added. The modified GLP-1 with the extra amino acid can then be fused to transferrin to make a fusion protein. In one embodiment, the GLP-1 peptide is modified to contain at least one additional amino acid at its amino terminus. In another embodiment, the GLP-1 peptide is modified to contain at least five additional amino acids at its amino terminus. Alternatively, the GLP-1 peptide is modified to contain between one and five additional amino acids at its amino terminus.

Glucagon-Like Peptide-1 (GLP-1) is a gastrointestinal hormone that regulates insulin secretion belonging to the so-called enteroinsular axis. The enteroinsular axis designates a group of hormones, released from the gastrointestinal mucosa in response to the presence and absorption of nutrients in the gut, which promote an early and potentiated release of insulin. The incretin effect which is the enhancing effect on insulin secretion is probably essential for a normal glucose tolerance. GLP-1 is a physiologically important insulinotropic hormone because it is responsible for the incretin effect.

GLP-1 is a product of proglucagon (Bell, et al., Nature, 1983, 304: 368-371). It is synthesized in intestinal endocrine cells in two principal major molecular forms, as GLP-1(7-36)amide and GLP-1(7-37). The peptide was first identified following the cloning of cDNAs and genes for proglucagon in the early 1980s.

Initial studies done on the full length peptide GLP-1(1-37 and 1-36^{amide}) concluded that the larger GLP-1 molecules are devoid of biological activity. In 1987, three independent research groups demonstrated that removal of the first six amino acids resulted in a GLP-1 molecule with enhanced biological activity.

The amino acid sequence of GLP-1 is disclosed by Schmidt et al. (1985 Diabetologia 28 704-707). Human GLP-1 is a 37 amino acid residue peptide originating from preproglucagon which is synthesized in the L-cells in the distal ileum, in the pancreas, and in the brain. Processing of preproglucagon to GLP-1(7-36)amide, GLP-1(7-37) and GLP-2 occurs mainly in the L-cells. The amino acid sequence of GLP-1(7-37) is SEQ ID NO: 32 (X=Gly):

His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly.

In GLP-1(7-36)amide, the terminal Gly is replaced by NH₂.

GLP-1 like molecules possesses anti-diabetic activity in human subjects suffering from Type II (non-insulin-dependent diabetes mellitus (NIDDM)) and, in some cases, even Type I diabetes. Treatment with GLP-1 elicits activity, such as increased insulin secretion and biosynthesis, reduced glucagon secretion, delayed gastric emptying, only at elevated glucose levels, and thus provides a potentially much safer therapy than insulin or sulfonylureas. Post-prandial and glucose levels in patients can be moved toward normal levels with proper GLP-1 therapy. There are also reports suggesting GLP-1-like molecules possess the ability to preserve and even restore pancreatic beta cell function in Type-II patients.

Any GLP-1 sequence may be modified by adding one or more amino acids at its amino terminus, including GLP-1(7-34), GLP-1(7-35), GLP-1(7-36), and GLP-1(7-37). GLP-1 also has powerful actions on the gastrointestinal tract. Infused in physiological amounts, GLP-1 potently inhibits pentagastrin-induced as well as meal-induced gastric acid secretion (Schjoldager et al., Dig. Dis. Sci. 1989, 35:703-708; Wettergren et al., Dig Dis Sci 1993; 38:665-673). It also inhibits gastric emptying rate and pancreatic enzyme secretion (Wettergren et al., Dig Dis Sci 1993; 38:665-673). Similar inhibitory effects on gastric and pancreatic secretion and motility may be elicited in humans upon perfusion of the ileum with carbohydrate- or lipid-containing solutions (Layer et al., Dig Dis Sci 1995, 40:1074-1082; Layer et al., Digestion 1993, 54: 385-38). Concomitantly, GLP-1 secretion is greatly stimulated, and it has been speculated that GLP-1 may be at least partly responsible for this so-called “ileal-brake” effect (Layer et al., Digestion 1993; 54: 385-38). In fact, recent studies suggest that, physiologically, the ileal-brake effects of GLP-1 may be more important than its effects on the pancreatic islets. Thus, in dose response studies GLP-1 influences gastric emptying rate at infusion rates at least as low as those required to influence islet secretion (Nauck et al., Gut 1995; 37 (suppl. 2): A124).

GLP-1 seems to have an effect on food intake. Intraventricular administration of GLP-1 profoundly inhibits food intake in rats (Schick et al. in Ditschuneit et al. (eds.), Obesity in Europe, John Libbey & Company ltd, 1994; pp. 363-367; Turton et al., Nature 1996, 379: 69-72). This effect seems to be highly specific. Thus, N-terminally extended GLP-1(1-36^{amide}) is inactive and appropriate doses of the GLP-1 antagonist, exendin 9-39, abolish the effects of GLP-1(Tang-Christensen et al., Am. J. Physiol., 1996, 271(4 Pt 2):R848-56). Acute, peripheral administration of GLP-1 does not inhibit food intake acutely in rats (Tang-Christensen et al., Am. J. Physiol., 1996, 271(4 Pt 2):R848-56; Turton et al., Nature 1996, 379: 69-72). However, it remains possible that GLP-1 secreted from the intestinal L-cells may also act as a satiety signal.

In diabetic patients, GLP-1's insulinotropic effects and the effects of GLP-1 on the gastrointestinal tract are preserved (Willms et al, Diabetologia 1994; 37, suppl. 1: A118), which may help curtail meal-induced glucose excursions, but, more importantly, may also influence food intake. Administered intravenously, continuously for one week, GLP-1 at 4 ng/kg/min has been demonstrated to dramatically improve glycaemic control in NIDDM patients without significant side effects (Larsen et al., Diabetes 1996; 45, suppl. 2: 233A.).

Modified GLP-1 partially or substantially protected from DPP activity and modified GLP-1 analogs are useful in the treatment of Type 1 and Type 2 diabetes and obesity.

As used herein, the term “GLP-1 molecule” means GLP-1, a GLP-1 analog, or GLP-1 derivative.

As used herein, the term “GLP-1 analog” is defined as a molecule having one or more amino acid substitutions, deletions, inversions, or additions compared with GLP-1. Many GLP-1 analogs are known in the art and include, for example, GLP-1(7-34), GLP-1(7-35), GLP-1(7-36), Val⁸-GLP-1(7-37), Gln⁹-GLP1(7-37), D-Gln⁹-GLP-1(7-37), Thr¹⁶-Lys¹⁸-GLP-1(7-37), and Lys¹⁸-GLP-1(7-37) (SEQ ID NO: 72). U.S. Pat. No. 5,118,666 discloses examples of GLP-1 analogs such as GLP-1(7-34) and GLP-1(7-35).

The term “GLP-1 derivative” is defined as a molecule having the amino acid sequence of GLP-1 or a GLP-1 analog, but additionally having chemical modification of one or more of its amino acid side groups, α-carbon atoms, terminal amino group, or terminal carboxylic acid group. A chemical modification includes, but is not limited to, adding chemical moieties, creating new bonds, and removing chemical moieties.

As used herein, the term “GLP-1 related compound” refers to any compound falling within the GLP-1, GLP-1 analog, or GLP-1 derivative definition.

WO 91/11457 discloses analogs of the active GLP-1 peptides 7-34,7-35, 7-36, and 7-37 which can also be useful as GLP-1 moieties.

EP 0708179-A2 (Eli Lilly & Co.) discloses GLP-1 analogs and derivatives that include an N-terminal imidazole group and optionally an unbranched C₆-C₁₀ acyl group in attached to the lysine residue in position 34.

EP 0699686-A2 (Eli Lilly & Co.) discloses certain N-terminal truncated fragments of GLP-1 that are reported to be biologically active.

U.S. Pat. No. 5,545,618 discloses GLP-1 molecules consisting essentially of GLP-1(7-34), GLP1(7-35), GLP-1(7-36), or GLP-1(7-37), or the amide forms thereof, and pharmaceutically-acceptable salts thereof, having at least one modification selected from the group consisting of: (a) substitution of glycine,serine, cysteine, threonine, asparagine, glutamine, tyrosine, alanine, valine, isoleucine, leucine, methionine, phenylalanine, arginine, or D-lysine for lysine at position 26 and/or position 34; or substitution of glycine, serine, cysteine, threonine, asparagine, glutamine, tyrosine, alanine, valine, isoleucine, leucine, methionine, phenylalanine, lysine, or a D-arginine for arginine at position 36 (SEQ ID NO: 73); (b) substitution of an oxidation-resistant amino acid for tryptophan at position 31 (SEQ ID NO: 74); (c) substitution of at least one of: tyrosine for valine at position 16; lysine for serine at position 18; aspartic acid for glutamic acid at position 21; serine for glycine at position 22; arginine for glutamine at position 23; arginine for alanine at position 24; and glutamine for lysine at position 26 (SEQ ID NO: 75); and (d) substitution of at least one of: glycine, serine, or cysteine for alanine at position 8; aspartic acid, glycine, serine, cysteine, threonine, asparagine, glutamine, tyrosine, alanine, valine, isoleucine, leucine, methionine, or phenylalanine for glutamic acid at position 9; serine, cysteine, threonine, asparagine, glutamine, tyrosine, alanine, valine, isoleucine, leucine, methionine, or phenylalanine for glycine at position 10; and glutamic acid for aspartic acid at position 15 (SEQ ID NO: 76); and (e) substitution of glycine, serine, cysteine, threonine, asparagine, glutamine, tyrosine, alanine, valine, isoleucine, leucine, methionine, or phenylalanine, or the D- or N-acylated or alkylated form of histidine for histidine at position 7 (SEQ ID NO: 77); wherein, in the substitutions is (a), (b), (d), and (e), the substituted amino acids can optionally be in the D-form and the amino acids substituted at position 7 can optionally be in the N-acylated or N-alkylated form.

U.S. Pat. No. 5,118,666 discloses a GLP-1 molecule having insulinotropic activity. Such molecule is selected from the group consisting of a peptide having the amino acid sequence His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys (GLP-1, 7-34, see SEQ ID NO: 32) or His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly (GLP-1, 7-35, see SEQ ID NO: 32); and a derivative of said peptide and wherein said peptide is selected from the group consisting of: a pharmaceutically-acceptable acid addition salt of said peptide; a pharmaceutically-acceptable carboxylate salt of said peptide; a pharmaceutically-acceptable lower alkylester of said peptide; and a pharmaceutically-acceptable amide of said peptide selected from the group consisting of amide, lower alkyl amide, and lower dialkyl amide.

U.S. Pat. No. 6,277,819 teaches a method of reducing mortality and morbidity after myocardial infarction comprising administering GLP-1, GLP-1 analogs, and GLP-1 derivatives to the patient. The GLP-1 analog being represented by the following structural formula (SEQ ID NO: **): R₁-X₁-Glu-Gly-Thr-Phe-Thr-Ser-Asp-Val-Ser-Ser-Tyr-Leu-X₂-Gly-Gin-Ala-Ala-Lys-X₃-Phe-Ile-Ala-Trp-Leu-Val-Lys-Gly-Arg-R₂ (SEQ ID NO: 78) and pharmaceutically-acceptable salts thereof, wherein: R₁ is selected from the group consisting of L-histidine, D-histidine, desamino-histidine, 2-amino-histidine, .beta.-hydroxy-histidine, homohistidine, alpha-fluoromethyl-histidine, and alpha-methyl-histidine; X₁ is selected from the group consisting of Ala, Gly, Val, Thr, Ile, and alpha-methyl-Ala; X₂ is selected from the group consisting of Glu, Gln, Ala, Thr, Ser, and Gly; X₃ is selected from the group consisting of Glu, Gln, Ala, Thr, Ser, and Gly; R₂ is selected from the group consisting of NH₂, and Gly—OH; provided that the GLP-1 analog has an isoelectric point in the range from about 6.0 to about 9.0 and further providing that when R₁ is His, X₁ is Ala, X₂ is Glu, and X₃ is Glu, R₂ must be NH₂.

Ritzel et al. (Journal of Endocrinology, 1998, 159: 93-102) disclose a GLP-1 analog, [Ser⁸]GLP-1, in which the second N-terminal alanine is replaced with serine. The modification did not impair the insulinotropic action of the peptide but produced an analog with increased plasma stability as compared to GLP-1.

U.S. Pat. No. 6,429,197 teaches that GLP-1 treatment after acute stroke or hemorrhage, preferably intravenous administration, can be an ideal treatment because it provides a means for optimizing insulin secretion, increasing brain anabolism, enhancing insulin effectiveness by suppressing glucagon, and maintaining euglycemia or mild hypoglycemia with no risk of severe hypoglycemia or other adverse side effects. The present invention provides a method of treating the ischemic or reperfused brain with GLP-1 or its biologically active analogues after acute stroke or hemorrhage to optimize insulin secretion, to enhance insulin effectiveness by suppressing glucagon antagonism, and to maintain euglycemia or mild hypoglycemia with no risk of severe hypoglycemia.

U.S. Pat. No. 6,277,819 provides a method of reducing mortality and morbidity after myocardial infarction, comprising administering to a patient in need thereof, a compound selected from the group consisting of GLP-1, GLP-1 analogs, GLP-1 derivatives and pharmaceutically-acceptable salts thereof, at a dose effective to normalize blood glucose.

U.S. Pat. No. 6,191,102 discloses a method of reducing body weight in a subject in need of body weight reduction by administering to the subject a composition comprising a glucagon-like peptide-1 (GLP-1), a glucagon-like peptide analog (GLP-1 analog), a glucagon-like peptide derivative (GLP-1 derivative) or a pharmaceutically acceptable salt thereof in a dose sufficient to cause reduction in body weight for a period of time effective to produce weight loss, said time being at least 4 weeks.

GLP-1 is fully active after subcutaneous administration (Ritzel et al., Diabetologia 1995; 38: 720-725), but is rapidly degraded mainly due to degradation by dipeptidyl peptidase IV-like enzymes (Deacon et al., J Clin Endocrinol Metab 1995, 80: 952-957; Deacon et al.,1995, Diabetes 44:1126-1131). Thus, unfortunately, GLP-1 and many of its analogues have a short plasma half-life in humans (Orskov et al., Diabetes 1993; 42:658-661). Accordingly, it is an objective of the present invention to provide modified GLP-1 or analogues thereof which have a protracted profile of action relative to GLP-1(7-37). It is a further object of the invention to provide derivatives of GLP-1 and analogues thereof which have a lower clearance than GLP-1(7-37). Moreover, it is an object of the invention to provide pharmaceutical compositions comprising modified GLP-1 or GLP-1 analogs with improved stability. Additionally, the present invention includes the use of modified GLP-1 or GLP-1 analogs to treat diseases associated with GLP-1 such as but not limited to those described above.

In one aspect of the present invention, the pharmaceutical compositions comprising modified GLP-1 and GLP-1 analogs may be formulated by any of the established methods of formulating pharmaceutical compositions, e.g. as described in Remington's Pharmaceutical Sciences, 1985. The composition may be in a form suited for systemic injection or infusion and may, as such, be formulated with a suitable liquid vehicle such as sterile water or an isotonic saline or glucose solution. The compositions may be sterilized by conventional sterilization techniques which are well known in the art. The resulting aqueous solutions may be packaged for use or filtered under aseptic conditions and lyophilized, the lyophilized preparation being combined with the sterile aqueous solution prior to administration. The composition may contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

The modified GLP-1 and GLP-1 analogs of the present invention may also be adapted for nasal, transdermal, pulmonal or rectal administration. The pharmaceutically acceptable carrier or diluent employed in the composition may be any conventional solid carrier. Examples of solid carriers are lactose, terra alba, sucrose, talc, gelatin, agar, pectin, acacia, magnesium stearate and stearic acid. Similarly, the carrier or diluent may include any sustained release material known in the art, such as glyceryl monostearate or glyceryl distearate, alone or mixed with a wax.

It may be of particular advantage to provide the composition of the invention in the form of a sustained release formulation. As such, the composition may be formulated as microcapsules or microparticles containing the modified GLP-1 or GLP-1 analogs encapsulated by or dispersed in a suitable pharmaceutically acceptable biodegradable polymer such as polylactic acid, polyglycolic acid or a lactic acid/glycolic acid copolymer.

For nasal administration, the preparation may contain modified GLP-1 or GLP-1 analogs dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

Generally, the modified polypeptides or peptides of the present invention are dispensed in unit dosage form together with a pharmaceutically acceptable carrier per unit dosage.

Moreover, the present invention contemplates the use of the modified GLP-1 and GLP-1 analogs for the manufacture of a medicinal product which can be used in the treatment of diseases associated with elevated glucose level (metabolic disease), such as but not to limited to those described above. Specifically, the present invention contemplates the use of modified GLP-1 and GLP-1 analogs for the treatment of diabetes including type II diabetes, obesity, severe burns, and heart failure, including congestive heart failure and acute coronary syndrome.

The present invention also provides modified Exendin-3 and Exendin-4 peptides partially and substantially protected from DPP activity. Exendin-3 and Exendin-4 are insulinotropic peptides comprising 39 amino acids (differing at residues 2 and 3) which are approximately 53% homologous to GLP-1. The Exendin-3 sequence is HSDGTFTSDLSKQMEEEAVRLFIEWLKNGG PSSGAPPPS (SEQ ID NO: 79), and the Exendin-4 sequence is HGEGTFTSDLSKQMEEEAVRLFEWLKNGG PSSGAPPPS (SEQ ID NO: 80). The invention also encompasses the modified exendin-4 fragments comprising the amino acid sequences such as Exendin-4 (1-31) HGEGTFTSDLSKQMEEAVR LFIEWLKNGGPY (SEQ ID NO: 81). Additionally, the present invention includes modified analogs of Exendin-3 and Exendin-4 peptides.

Modified GLP-1 Fusion Protein or Conjugate for Treating Type 2 Diabetes

The modified GLP-1 may be fused to a heterologous molecule for increased overall stability in vivo. The modified GLP-1 may be fused to a heterologous molecule by recombinant means or covalently attached to a heterologous molecule by methods well known in the art. Modified GLP-1 may be fused or covalently attached, for example to a plasma protein such as serum albumin or transferrin, an immunoglobulin, or a portion thereof such as the Fc domain. More preferably, the modified polypeptide or peptide is fused to transferrin, lactotransferrin, or melanotransferrin. Methods for making such fusion proteins are provided by U.S. application Ser. No. 10/378,094, which is herein incorporated by reference in its entirety.

The GLP-1 molecule may be attached to the heterologous protein via a linker of variable length to provide greater physical separation and allow more spatial mobility between the fused proteins and thus maximize the accessibility of the therapeutic protein, for instance, for binding to its cognate receptor. The linker peptide may consist of amino acids that are flexible or more rigid. For example, a linker such as a poly-glycine stretch may be used. The linker can be less than about 50, 40, 30, 20, 10, or 5 amino acid residues. The linker can be covalently linked to and between the heterologous protein and GLP-1. Preferably, the linker may be one Ser residue, two Ser residues, or the peptide Ser-Ser-Gly. These linkers may be used to link GLP-1 to transferrin.

The transferrin to be attached to the modified polypeptide or peptide may be modified. It may exhibit reduced glycosylation. The modified transferrin polypeptide may be selected from the group consisting of a single transferrin N domain, a single transferrin C domain, a transferrin N and C doman, two transferrin N domains, and two transferrin C domains.

As discussed above, GLP-1 activates and regulates important endocrine hormone systems in the body and plays a critical management role in the metabolism of glucose. Unlike all other diabetic treatments on the market GLP-1 has the potential to be restorative by acting as a growth factor for β-cells thus improving the ability of the pancreas to secrete insulin and also, to make the existing insulin levels act more efficiently by improving sensitivity and better stabilizing glucose levels. This reduces the burden on daily monitoring of glucose levels and potentially offers a delay in the serious long term side effects caused by fluctuations in blood glucose due to diabetes. Furthermore, GLP-1 can reduce appetite and reduce weight. Obesity is an inherent consequence of poor control of glucose metabolism and this only serves to aggravate the diabetic condition.

Clinical application of natural GLP-1 is limited because it is rapidly degraded in the circulation (half-life is several minutes). To maintain therapeutic levels in the circulation requires constant administration of high doses using pumps or patch devices which adds to the cost of treatment. This is inconvenient for long term chronic use especially in conjunction with all the other medications for treating diabetes and monitoring of glucose levels. The modified GLP-1 fusion proteins retain the activity of GLP-1 but have the long half-life (14-17 days), solubility, and biodistribution properties of transferrin. These properties could provide for a low cost, small volume, monthly s.c. (subcutaneous) injection and this type of product is absolutely needed for long term chronic use.

The modified GLP-1 also may be covalently attached to a blood component to increase its stability. For example, the modified GLP-1 may be covalently attached to serum albumin, transferrin, immunoglobulin, or the Fc portion of the immunoglobulin. In one embodiment, the modified GLP-1 may be attached to a fatty acid or a fatty acid derivative. In another embodiment, the modified GLP-1 may be engineered into a drug affinity complex (DAC). As discussed earlier, Kim et al. (2003, Diabetes 52(3):751) disclose a GLP-1-albumin drug affinity complex. Kim et al. show that the albumin-conjugated DAC:GLP-1 mimics the native GLP-1. Kim et al. provide a new approach for prolonged activation of GLP-LR signaling.

Upon subcutaneous administration, the DAC:modified GLP-1 rapidly and selectively bonds in vivo to albumin. The bioconjugate formed has the same therapeutic activity and similar potency as endogenous GLP-1 but has a pharmacokinetic profile that is closer to albumin.

Modified GLP-1 and its Fusion Protein in Combination with Other Therapeutic Agents

In one aspect of the invention, the modified GLP-1 peptide and its fusion protein, for example, GLP/mTf fusion protein, of the present invention are used in combination with at least one second therapeutic molecule such as Glucophage® (metformin hydrochloride tablets) or Glucophage® XR (metformin hydrochloride extended-release tablets) to treat type II diabetes, obesity, and other diseases or conditions associated with abnormal glucose levels.

Glucophage® and Glucophage® XR are oral antihyperglycemic drugs for the management of type II diabetes. Glucophage® XR is an extended release formulation of Glucophage. Accordingly, Glucophage® XR may be taken once daily because the drug is released slowly from the dosage form. Glucophage® helps the body produce less glucose from the liver. Accordingly, Glucophage® is effective in controlling blood sugar level in a patient. Glucophage® rarely causes low blood glucose (hypoglycemia) because it does not cause the body to make more insulin.

Glucophage® also helps lower the fatty blood components, triglycerides and cholesterol, that are often high in people with Type II diabetes. Metformin has been shown to decrease the appetite and help people lose a few pounds when they starting taking the medicine.

Metformin has been approved for treatment with sulfonylureas, or with insulin, or as monotherapy (by itself). Metformin has been suggested for use in treating various cardiovascular diseases such as hypertension in insulin resistant patients (WO 9112003-Upjohn), for dissolving blood clots (in combination with a t-PA-derivative) (WO 9108763, WO 9108766, WO 9108767 and WO 9108765-Boehringer Mannheim), ischemia and tissue anoxia (EP 283369-Lipha), atherosclerosis (DE 1936274-Brunnengraber & Co., DE 2357875-Hurka, and U.S. Pat. No. 4,205,087-ICI). In addition, it has been suggested to use metformin in combination with prostaglandin-analogous cyclopentane derivatives as coronary dilators and for blood pressure lowering (U.S. Pat. No. 4,182,772-Hoechst). Metformin has also been suggested for use in cholesterol lowering when used in combination with 2-hydroxy-3,3,3-trifluoropropionic acid derivatives (U.S. Pat. No. 4,107,329-ICI), 1,2-diarylethylene derivatives (U.S. Pat. No. 4,061,772-Hoechst), substituted aryloxy-3,3,3-trifluoro-2-propionic acids, esters and salts (U.S. Pat. No. 4,055,595-ICI), substituted hydroxyphenyl-piperidones (U.S. Pat. No. 4,024,267-Hoechst), and partially hydrogenated 1H-indeno-[1,2B]-pyridine derivatives (U.S. Pat. No. 3,980,656-Hoechst).

Montanari et al. (Pharmacological Research, Vol. 25, No. 1, 1992) disclose that use of metformin in amounts of 500 mg twice a day (b.i.d.) increased post-ischemia blood flow in a manner similar to 850 mg metformin three times a day (t.i.d.). Sirtori et al. (J. Cardiovas. Pharm., 6:914-923, 1984), disclose that metformin in amounts of 850 mg three times a day (t.i.d) increased arterial flow in patients with peripheral vascular disease.

The present invention provides the treatment of various diseases comprising modified GLP-1 of the present invention or its fusion protein in combination with one or more therapeutic agents such as metformin. In one embodiment, the modified GLP-1 or its fusion protein in combination with metformin is used to treat diseases and conditions associated with abnormal blood glucose level, such as diabetes. Preferably, the GLP-1/mTf fusion protein in combination with metformin is used to treat type II diabetes or obesity.

Other therapeutic agents that may be used in combination with modified GLP-1 of the present invention and its fusion proteins include but are not limited to sulfonylurea and sulfonylurea-like agents, thiazolidinediones, Peroxisome Proliferator-Activated Receptor (PPAR) gamma modulators, PPAR alpha modulators, Protein Tyrosine Phosphatase-1B inhibitors, Insulin Receptor Tyrosine Kinase activators, 11beta-hydroxysteroid dehydrogenase inhibitors, glycogen phosphorylase inhibitors, glucokinase activators, beta-3 adrenergic agonists, and glucagon receptor agonists.

Transgenic Animals

The production of transgenic non-human animals that express a modified polypeptide or peptide that is protected from DPP activity is contemplated in one embodiment of the present invention. In some embodiments, transgenic non-human animals expressing fusion proteins comprising a modified polypeptide or peptide and having increased stability is contemplated.

The successful production of transgenic, non-human animals has been described in a number of patents and publications, such as, for example U.S. Pat. No. 6,291,740 (issued Sep. 18, 2001); U.S. Pat. No. 6,281,408 (issued Aug. 28, 2001); and U.S. Pat. No. 6,271,436 (issued Aug. 7, 2001) the contents of which are hereby incorporated by reference in their entireties.

The ability to alter the genetic make-up of animals, such as domesticated mammals including cows, pigs, goats, horses, cattle, and sheep, allows a number of commercial applications. These applications include the production of animals which express large quantities of exogenous proteins in an easily harvested form (e.g., expression into the milk or blood), the production of animals with increased weight gain, feed efficiency, carcass composition, milk production or content, disease resistance and resistance to infection by specific microorganisms and the production of animals having enhanced growth rates or reproductive performance. Animals which contain exogenous DNA sequences in their genome are referred to as transgenic animals.

The most widely used method for the production of transgenic animals is the microinjection of DNA into the pronuclei of fertilized embryos (Wall et al., J. Cell. Biochem. 49:113 [1992]). Other methods for the production of transgenic animals include the infection of embryos with retroviruses or with retroviral vectors. Infection of both pre- and post-implantation mouse embryos with either wild-type or recombinant retroviruses has been reported (Janenich, Proc. Natl. Acad. Sci. USA 73:1260 [1976]; Janenich et al., Cell 24:519 [1981]; Stuhlmann et al., Proc. Natl. Acad. Sci. USA 81:7151 [1984]; Jahner et al., Proc. Natl. Acad Sci. USA 82:6927 [1985]; Van der Putten et al., Proc. Natl. Acad Sci. USA 82:6148-6152 [1985]; Stewart et al., EMBO J. 6:383-388 [1987]).

An alternative means for infecting embryos with retroviruses is the injection of virus or virus-producing cells into the blastocoele of mouse embryos (Jahner, D. et al., Nature 298:623 [1982]). The introduction of transgenes into the germline of mice has been reported using intrauterine retroviral infection of the midgestation mouse embryo (Jahner et al., supra [1982]). Infection of bovine and ovine embryos with retroviruses or retroviral vectors to create transgenic animals has been reported. These protocols involve the micro-injection of retroviral particles or growth arrested (i.e., mitomycin C-treated) cells which shed retroviral particles into the perivitelline space of fertilized eggs or early embryos (PCT International Application WO 90/08832 [1990]; and Haskell and Bowen, Mol. Reprod. Dev., 40:386 [1995]. PCT International Application WO 90/08832 describes the injection of wild-type feline leukemia virus B into the perivitelline space of sheep embryos at the 2 to 8 cell stage. Fetuses derived from injected embryos were shown to contain multiple sites of integration.

U.S. Pat. No. 6,291,740 (issued Sep. 18, 2001) describes the production of transgenic animals by the introduction of exogenous DNA into pre-maturation oocytes and mature, unfertilized oocytes (i.e., pre-fertilization oocytes) using retroviral vectors which transduce dividing cells (e.g., vectors derived from murine leukemia virus [MLV]). This patent also describes methods and compositions for cytomegalovirus promoter-driven, as well as mouse mammary tumor LTR expression of various recombinant proteins.

U.S. Pat. No. 6,281,408 (issued Aug. 28, 2001) describes methods for producing transgenic animals using embryonic stem cells. Briefly, the embryonic stem cells are used in a mixed cell co-culture with a morula to generate transgenic animals. Foreign genetic material is introduced into the embryonic stem cells prior to co-culturing by, for example, electroporation, microinjection or retroviral delivery. ES cells transfected in this manner are selected for integrations of the gene via a selection marker such as neomycin.

U.S. Pat. No. 6,271,436 (issued Aug. 7, 2001) describes the production of transgenic animals using methods including isolation of primordial germ cells, culturing these cells to produce primordial germ cell-derived cell lines, transforming both the primordial germ cells and the cultured cell lines, and using these transformed cells and cell lines to generate transgenic animals. The efficiency at which transgenic animals are generated is greatly increased, thereby allowing the use of homologous recombination in producing transgenic non-rodent animal species.

Gene Therapy

The use of modified polypeptide or peptide constructs of the present invention for gene therapy is contemplated in one embodiment of this invention. The polypeptide or peptide has been modified to protect it from DPP activity by the addition of one or more additional amino acids at its N-terminus. For example, the nucleic acid construct encoding GLP-1 comprising an additional His residue at its N-terminus is provided for gene therapy. Also, the nucleic acid construct encoding modified GLP-1/transferrin fusion protein is provided for gene therapy. The modified GLP-1 constructs of the present invention are protected from DPP activity and are more stable; thus, they are ideally suited to gene therapy treatments.

Briefly, gene therapy via injection of an adenovirus vector containing a gene encoding a soluble fusion protein consisting of cytotoxic lymphocyte antigen 4 (CTLA4) and the Fc portion of human immunoglubulin G1 was recently shown in Ijima et al. (Jun. 10, 2001) Human Gene Therapy (United States) 12/9:1063-77. In this application of gene therapy, a murine model of type II collagen-induced arthritis was successfully treated via intraarticular injection of the vector.

Gene therapy is also described in a number of U.S. patents including U.S. Pat. No. 6,225,290 (issued May 1, 2001); U.S. Pat. No. 6,187,305 (issued Feb. 13, 2001); and U.S. Pat. No. 6,140,111 (issued Oct. 31, 2000).

U.S. Pat. No. 6,225,290 provides methods and constructs whereby intestinal epithelial cells of a mammalian subject are genetically altered to operatively incorporate a gene which expresses a protein which has a desired therapeutic effect. Intestinal cell transformation is accomplished by administration of a formulation composed primarily of naked DNA, and the DNA may be administered orally. Oral or other intragastrointestinal routes of administration provide a simple method of administration, while the use of naked nucleic acid avoids the complications associated with use of viral vectors to accomplish gene therapy. The expressed protein is secreted directly into the gastrointestinal tract and/or blood stream to obtain therapeutic blood levels of the protein thereby treating the patient in need of the protein. The transformed intestinal epithelial cells provide short or long term therapeutic cures for diseases associated with a deficiency in a particular protein or which are amenable to treatment by overexpression of a protein.

U.S. Pat. No. 6,187,305 provides methods of gene or DNA targeting in cells of vertebrate, particularly mammalian, origin. Briefly, DNA is introduced into primary or secondary cells of vertebrate origin through homologous recombination or targeting of the DNA, which is introduced into genomic DNA of the primary or secondary cells at a preselected site.

U.S. Pat. No. 6,140,111 (issued Oct. 31, 2000) describes retroviral gene therapy vectors. The disclosed retroviral vectors include an insertion site for genes of interest and are capable of expressing high levels of the protein derived from the genes of interest in a wide variety of transfected cell types. Also disclosed are retroviral vectors lacking a selectable marker, thus rendering them suitable for human gene therapy in the treatment of a variety of disease states without the co-expression of a marker product, such as an antibiotic. These retroviral vectors are especially suited for use in certain packaging cell lines. The ability of retroviral vectors to insert into the genome of mammalian cells has made them particularly promising candidates for use in the genetic therapy of genetic diseases in humans and animals. Genetic therapy typically involves (1) adding new genetic material to patient cells in vivo, or (2) removing patient cells from the body, adding new genetic material to the cells and reintroducing them into the body, i.e., in vitro gene therapy. Discussions of how to perform gene therapy in a variety of cells using retroviral vectors can be found, for example, in U.S. Pat. Nos. 4,868,116, issued Sep. 19, 1989, and 4,980,286, issued Dec. 25, 1990 (epithelial cells), WO 89/07136 published Aug. 10, 1989 (hepatocyte cells), EP 378,576 published Jul. 25, 1990 (fibroblast cells), and WO 89/05345 published Jun. 15, 1989 and WO/90/06997, published Jun. 28, 1990 (endothelial cells), the disclosures of which are incorporated herein by reference.

Without further description, it is believed that one of ordinary skill in the art can, using the preceding description and the following illustrative examples, make and utilize the claimed invention. The following working examples therefore, specifically point out preferred embodiments of the present invention, and are not to be construed as limiting in any way the remainder of the disclosure. All articles, publications, patents and documents referred to throughout this application are hereby incorporated by reference in their entirety.

### EXAMPLES

Example 1

### Modified GLP-1 Having Dipeptidyl-peptidase IV Protection

This Example describes modified GLP-1 peptides protected from DPPIV activity. The following peptides were synthesized using standard solid phase Fmoc chemistry and purified by reverse phase HPLC using a C18 column and quantitated by absorbance at 220 nm. The purified peptides were analyzed by mass spectrometry (MALDI-TOF):

GLP-1

(amino acids 1-30 of SEQ ID NO: 32)

NH₂-His-Ala-Glu-Gly-Thr-Phe-Thr-Ser-Asp-Val-Ser-
Ser-Tyr-Leu-Glu-Gly-Gln-Ala-Ala-Lys-Glu-Phe-Ile-
Ala-Trp-Leu-Val-Lys-Gly-Arg-COOH
GLP-1 (A8G)

(SEQ ID NO: 90)

NH₂-His-Gly-Glu-Gly-Thr-Phe-Thr-Ser-Asp-Val-Ser-
Ser-Tyr-Leu-Glu-Gly-Gln-Ala-Ala-Lys-Glu-Phe-Ile-
Ala-Trp-Leu-Val-Lys-Gly-Arg-COOH
H-GLP-1

(SEQ ID NO: 91)

NH₂-His-His-Ala-Glu-Gly-Thr-Phe-Thr-Ser-Asp-Val-
Ser-Ser-Tyr-Leu-Glu-Gly-Gln-Ala-Ala-Lys-Glu-Phe-
Ile-Ala-Trp-Leu-Val-Lys-Gly-Arg-COOH
H-GLP-1 (A8G)

(SEQ ID NO: 92)

NH₂-His-His-Gly-Glu-Gly-Thr-Phe-Thr-Ser-Asp-Val-
Ser-Ser-Tyr-Leu-Glu-Gly-Gln-Ala-Ala-Lys-Glu-Phe-
Ile-Ala-Trp-Leu-Val-Lys-Gly-Arg-COOH
HH-GLP-1

(SEQ ID NO: 93)

NH₂-His-His-His-Ala-Glu-Gly-Thr-Phe-Thr-Ser-Asp-
Val-Ser-Ser-Tyr-Leu-Glu-Gly-Gln-Ala-Ala-Lys-Glu-
Phe-Ile-Ala-Trp-Leu-Val-Lys-Gly-Arg-COOH
G-GLP-1

(SEQ ID NO: 94)

NH₂-Gly-His-Ala-Glu-Gly-Thr-Phe-Thr-Ser-Asp-Val-
Ser-Ser-Tyr-Leu-Glu-Gly-Gln-Ala-Ala-Lys-Glu-Phe-
Ile-Ala-Trp-Leu-Val-Lys-Gly-Arg-COOH
H-Exendin-4

(SEQ ID NO: 95)

NH₂-His-His-Gly-Glu-Gly-Thr-Phe-Thr-Ser-Asp-Leu-
Ser-Lys-Gln-Met-Glu-Glu-Glu-Ala-Val-Arg-Leu-Phe-
Ile-Glu-Trp-Leu-Lys-Asn-Gly-Gly-Pro-Ser-Ser-Gly-
Ala-Pro-Pro-Pro-Ser-COOH

Dipeptidylpeptidase-IV Treatment

Equimolar concentrations of each peptide (6 μM) were treated with 2 μg of recombinant human DPP-IV (1 μg/μL, R&D Systems, Minneapolis, MN) in 25 mM Tris-Cl (pH 8.0). Control reactions excluding DPP-IV were set up in parallel for each peptide. The digests were incubated at room temperature for 2 hours, at which time the reactions were diluted 10-fold in Krebs-Ringer buffer (Biosource International, Camarillo, Calif.) supplemented with 1 mM 3-Isobutyl-1-methylxanthine (IBMX, Calbiochem, San Diego, Calif.). The peptides were then analyzed to determine residual GLP-1 receptor activating activity, as described below.

Cyclic AMP Stimulation Assay

Four 96-well tissue culture plates were seeded with CHO-GLP1R cells (Montrose-Rafizadeh, et al. 1997 J Biol. Chem. 272, 21201-21206) at a density of 2×10⁴ cells/well in RPMI/10% FBS medium one day prior to treatment. The next day the cells appeared uniformly distributed with an approximate confluency of 60-80 percent. One day after seeding the culture plates the cells were washed twice with Krebs-Ringer buffer (KRB) followed by incubation in KRB for 1 hr at 37° C. to lower the intracellular levels of cAMP. This was followed by incubation for 10 minutes in KRB/IBMX to inhibit intracellular enzymes that break down cAMP. Dilutions of each test compound were prepared in KRB/IBMX and triplicate wells of CHO-GLP1R cells were treated with 50 μl of test compound per well for exactly 20 minutes at 37° C. The treatment was halted by washing the cultures twice with ice-cold phosphate-buffered saline. Lysates were prepared by the addition of 0.1 ml lysis buffer 1B (Amersham Biosciences cAMP Biotrak EIA kit) for 10 minutes at room temperature. The entire volume of each cell extract was then assayed to determine the cAMP concentration using the cAMP Biotrak Enzyme Immunoassay System (Amersham Biosciences Corporation, Piscataway, N.J., product code RPN225) according to kit instructions. Peptides of the invention were found to be more resistant to DPP-IV than the unmodified forms.

Active GLP-1 specific ELISA

Alternatively, DPP-IV degradation of GLP-1 and GLP-1 derivatives of the invention was assayed using an ELISA system (Glucagon-Like Peptide-1 [Active] ELISA kit [Linco Research, Inc., St. Charles, Mo.]) that is specific for intact, active GLP-1 and does not recognize GLP-1 in which the N-terminal two amino acids have been removed due to the action of DPP-IV, i.e. GLP-1(9-36 or 9-37). Equimolar concentrations of GLP-1 and H-GLP-1 (1200 pM) were treated with recombinant human DPP-IV (200 ng/μL, R&D Systems, Minneapolis, Minn.) in 25 mM Tris-Cl (pH 8.0) and the reaction stopped by dilution in the assay buffer supplied with the kit, which contains protease inhibitors.

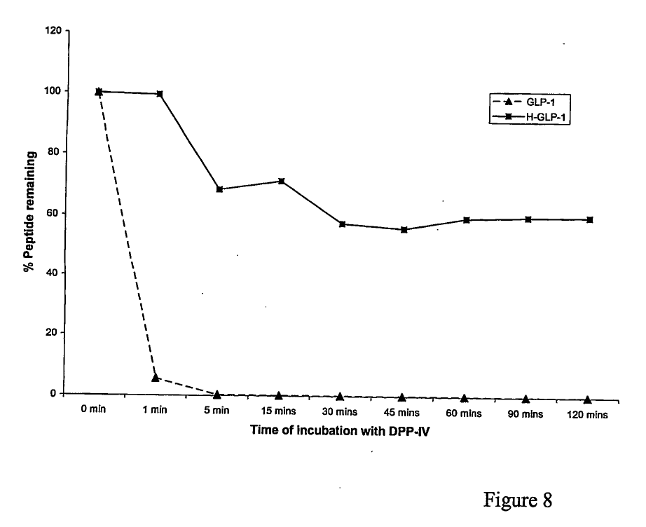

The kit comprises a 96-well microtitre plate coated with anti-GLP-1 monoclonal antibody. The plate was washed (25 mM Borate-buffered Saline x4 in a plate washer, ThermoLabsystems Ultrawash Plus), then incubated with peptide samples (300 pM and 10-fold serial dilutions down the plate) for 3 hours at room temperature. After washing as described above, the plate was incubated with Alkaline-Phosphatase-conjugated anti-GLP antibody (supplied as a ready-to-use component of the kit) for 2 hours at room temperature. After washing, 4-Methylumbelliferyl Phosphate (MUP) substrate (1:200 dilution in 50 mM Borate pH 9.5) was applied to all wells, and incubated in the dark at room temperature for 30 minutes. The plate was read at 355 excitation and 460 nm emission wavelengths on a SpectraMax Gemini EM fluorescence plate reader. As H-GLP-1 bound less readily to the monoclonal antibody than GLP-1 itself, the concentration of active H-GLP-1 remaining after DPP-IV treatment was determined using an H-GLP-1 standard curve. FIG. 8 shows that H-GLP-1 is substantially more resistant to the action of DPP-IV than GLP-1.

### Example 2

### Modified GLP-1 Fusion Protein

This Example describes a fusion protein comprising a modified GLP-1 protected from DPPIV activity fused to a modified transferrin molecule.

In order to construct a sequence encoding the transferrin secretion leader followed by GLP-1 and the N-terminal part of transferrin, the following overlapping primers were designed:

P0236-

(SEQ ID NO: 96)

TTCCCATACAAACTTAAGAGTCCAATTAGCTTCATCGCCA
P0237-

(SEQ ID NO: 97)

GGTTTAGCTTGTTTTTTTATTGGCGATGAAGCTAATTGGACTCTTAAGTT
TGTATGGGAA
P0244-

(SEQ ID NO: 98)

ATAAAAAAACAAGCTAAACCTAATTCTAACAAGCAAAGATGAGGCTCGCC
GTGGGAGCCC
P0245-

(SEQ ID NO: 99)

CAGGACGGCGCAGACCAGCAGGGCTCCCACGGCGAGCCTCATCTTTGCTT
GTTAGAATTA
P0248-

(SEQ ID NO: 100)

TGCTGGTCTGCGCCGTCCTGGGGCTGTGTCTGGCGCATGCTGAAGGTACT
TTTACTTCTGATGTTTCTTC
P0249-

(SEQ ID NO: 101)

AATTCTTTAGCAGCTTGACCTTCCAAATAAGAAGAAACATCAGAAGTAAA
AGTACCTTCAGCATGCGCCAGACACAGCCC
P0250-

(SEQ ID NO: 102)

TTATTTGGAAGGTCAAGCTGCTAAAGAATTTATTGCTTGGTTGGTTAAAG
GTAGGGTACCTGATAAAACT
P0251-

(SEQ ID NO: 103)

AGTTTTATCAGGTACCCTACCTTTAACCAACCAAGCAATA

The positions of these primers are shown below.

| | |
|---|---|
| | Af1II |
| | -−---- |
| | >>...........P0236............>> |
| 721 | ccaatgttac gtcccgttat attggagttc ttcccataca aacttaagag tccaattagc |
| | ggttacaatg cagggcaata taacctcaag aagggtatgt ttgaattctc aggttaatcg |
| | <<...........P0237............<< |
| | >>P0236.>> >>.....................P0244........................>> |
| 781 | ttcatcgcca ataaaaaaac aagctaaacc taattctaac aagcaaagat gaggctcgcc |
| | aagtagcggt tatttttttg ttcgatttgg attaagattg ttcgtttcta ctccgagcgg |
| | <<...........P0237............<< <<...........P0245............<< |
| | >>...nL.....> |
| | m r l a |
| | >>P0244.>> >>.....................P0248........................>> |
| 841 | gtgggagccc tgctggtctg cgccgtcctg gggctgtgtc tggcgcatgc tgaaggtact |
| | caccctcggg acgaccagac gcggcaggac Cccgacacag accgcgtacg acttccatga |
| | <<...........P0245............<< <<...........P0249............<< |
| | >......................nL......................>> |
| | v g a l l v c a v l g l c l a |
| | >>...GLP-1.....> |
| | h a e g t |
| | >>.....P0248.......>> >>...............P0250...................>> |
| 901 | tttacttctg atgtttcttc ttatttggaa ggtcaagctg ctaaagaatt tattgcttgg |
| | aaatgaagac tacaaagaag aataaacctt ccagttcgac gatttcttaa ataacgaacc |
| | <<...................P0249..........................<< <<.P0251<< |
| | >.............................GLP-1.............................> |
| | f t s d v s s y l e g q a a k e f i a w |
| | KpnI |
| | -----− |
| | >>.........P0250..............>> |
| 961 | ttggttaaag gtagggtacc tgataaaact gtgagatggt gtgcagtgtc ggagcatgag |
| | aaccaatttc catcccatgg actattttga cactctacca cacgtcacag cctcgtactc |
| | <<...........P0251............<< |
| | >....GLP-1....>> |
| | l v k g r |
| | >>.....................mTf......................> |
| | v p d k t v r w c a v s e h e |

(SEQ ID NO: 104 is the coding strand; SEQ ID NO: 105 is the encoded protein.)
The primers (8 μL of 20 pmol conc.) were combined and heated to 65° C. for 5 min. and then the annealing reaction was allowed to cool slowly to room temperature.

After adding T4 DNA ligase to the annealing reaction and incubating for a further 2 hr at room temperature, 1 μL of the reaction was removed and used in a PCR reaction to amplify the completed insert with the outer primers P0236 and P0251. The PCR conditions were as follows:
5 min at 94° C.
25 cycles of: 30 sec at 94° C.
   30 sec at 50° C.
   1 min at 72° C.
   7 min at 72° C.
   hold at 4° C.

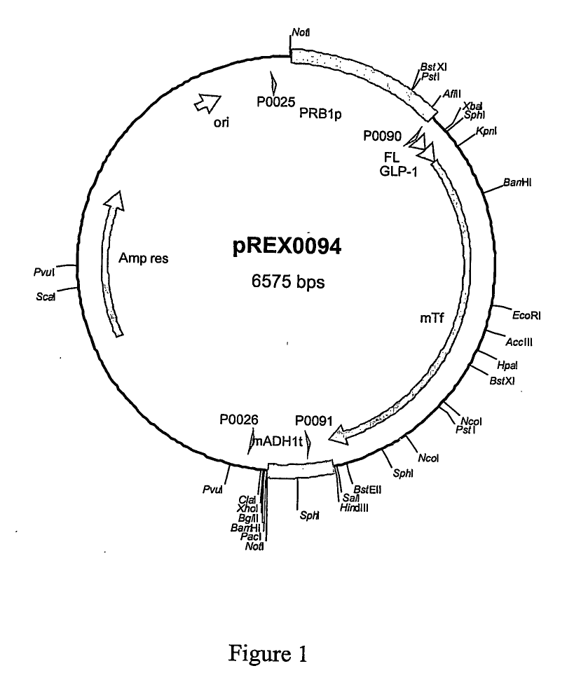

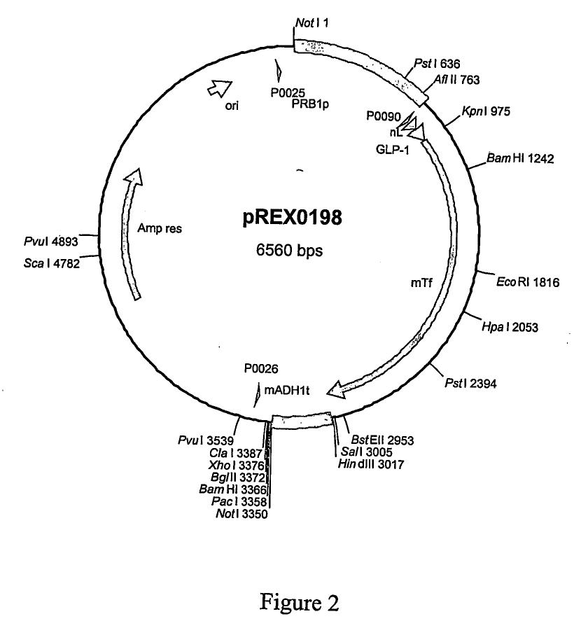

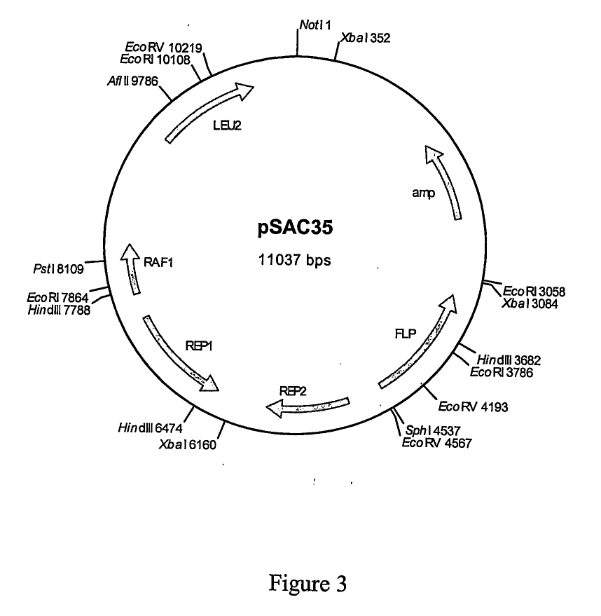

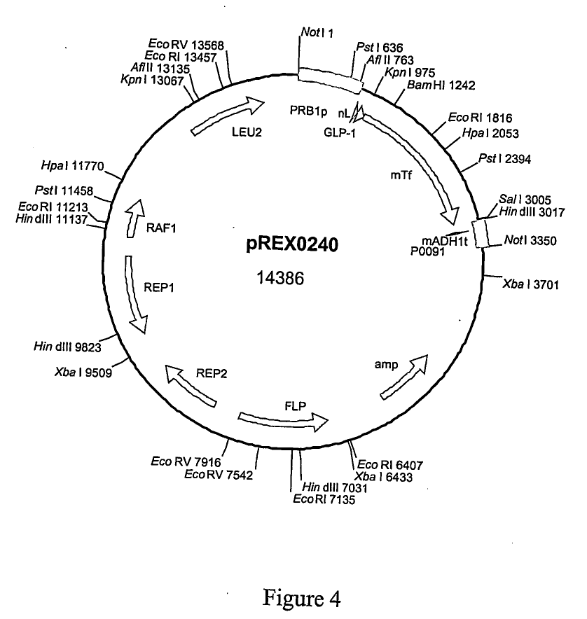

The resulting PCR product was digested AflII and KpnI and ligated into pREX0094 (FIG. 1) which had previously been digested with AflII and KpnI. The ligation was used to transform E. coli. The DNA from the resultant clones was sequenced and a clone correct the length of the AflII/KpnI insert was selected and designated pREX0198 (FIG. 2). Next, pREX0198 was digested with NotI and PvuI and inserted into pSAC35 (FIG. 3) to create pREX0240 (FIG. 4).

To create a plasmid encoding the natural transferrin secretion leader followed by H-GLP-1(7-36) fused to modified transerrin (mTf), overlapping primers P0424 and P0425 were designed to add the extra N-terminal histidine to the sequence encoded by pREX0198.

P0424 5′ to 3′

(SEQ ID NO: 106)

CTGTGTCTGGCGCATCATGCTGAAG
P0425 5′ to 3′

(SEQ ID NO: 107)

CTTCAGCATGATGCGCCAGACACAG

pREX0198 was used as the template for the initial PCR reactions using the two overlapping mutagenic primers and two outer primers in separate reactions, i.e. P0424 plus P0012 and P0425 plus P0025. The products of these reactions were then used as templates in a second round of PCR with just the outer primers, i.e. P0012 plus P0025, in order to join them together. The reaction conditions for both rounds of PCR were 1×94° C. for 1 min, 20×94° C. for 30 seconds, 50° C. for 30 seconds, 72° C. for 1 minute and 1×72° C. for 7 minutes to finish.

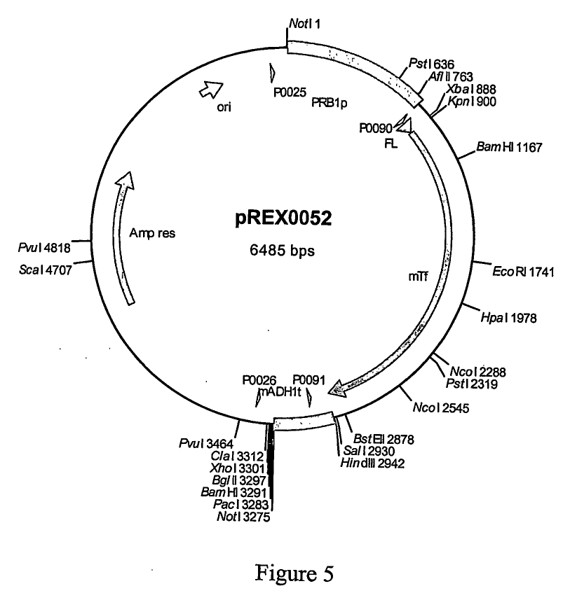

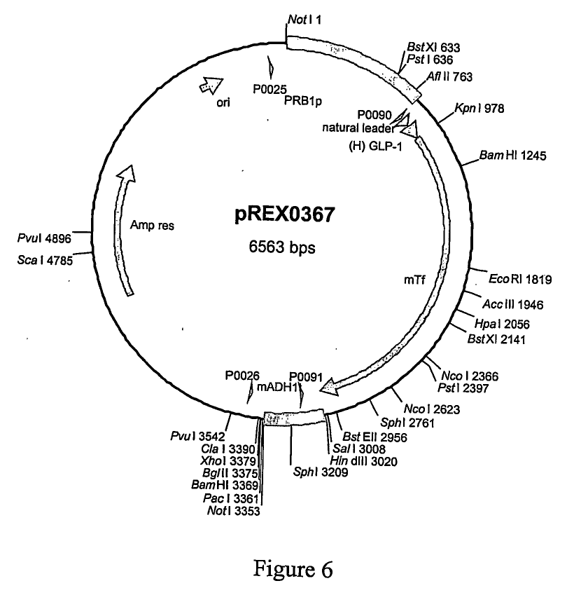

The PCR product from the final reaction was digested with AflII and KpnI and ligated into AflII/KpnI digested pREX0052 (FIG. 5) to create pREX0367 (FIG. 6). The construct was DNA sequenced to confirm the insertion of the codon for the extra histidine.

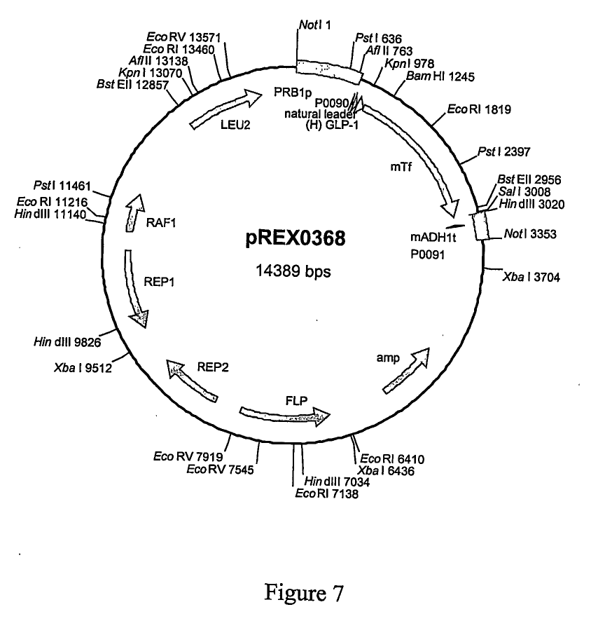

pREX0367 was then digested with NotI and PvuI (the latter to destroy the ampicillin resistance gene) and ligated into pSAC35 previously digested with NotI to create pREX0368 (FIG. 7).

pREX0368 was transformed into the host Saccharomyces cerevsiae strain by electroporation and transformed colonies selected on the basis of leucine prototrophy on buffered minimal medium plates. After selection of single colonies, yeast transformants were stocked in 40% Trehalose and stored at −70° C. Expression was determined by growth in liquid minimal medium buffered to pH 6.5 and analysis of supernatant by SDS-PAGE, western blot and ELISA.

The plasmids encoding GLP-1/mTf (PREX0100) and H-GLP-1/mTf were constructed as described in U.S. application Ser. No. 10/378,094, filed Mar. 4, 2003, which is herein incorporated by reference in its entirety. To produce the GLP-1/mTf fusion protein, the amino acid sequence of GLP-1(7-36) and GLP-1(7-37) may be used.

(amino acids 1-30 of SEQ ID NO: 32)

haegtftsdvssylegqaakefiawlvkgr

(SEQ ID NO: 32)

haegtftsdvssylegqaakefiawlvkgrg

For example, the peptide sequence of GLP-1(7-36) may be back translated into DNA and codon optimized for yeast:

catgctgaaggtacttttacttctgatgtttcttcttatttggaaggtcaagctgctaaagaa
h a e g t f t s d v s s y l e g q a a k e
tttattgcttggttggttaaaggtaga (SEQ ID NO: 117)
f i a w l v k g r (amino acids 1-30 of SEQ ID NO: 32)

The primers were specifically designed to form 5′ XbaI and 3′ KpnI sticky ends after annealing and to enable direct ligation into xbaI/KpnI cut pREX0052, just 5′ of the end of the leader sequence and at the N-terminus of mTf. Alternatively, other sticky ends may be engineered for ligations into other vectors.

| SEQ ID NOs: 118 and 119 | |
|---|---|
| | XbaI |
| | -+----- |
| 1 | aggtctctag agaaaaggca tgctgaaggt acttttactt ctgatgtttc ttcttatttg |
| | tccagagatc tcttttccgt acgacttcca tgaaaatgaa gactacaaag aagaataaac |
| | >>......FL.......>> |
| | r s l e k r |
| | >>..................GLP-1....................> |
| | h a e g t f t s d v s s y l |
| | KpnI |
| | ------+ |
| 61 | gaaggtcaag ctgctaaaga atttattgct tggttggtta aaggtagggt acctgata |
| | cttccagttc gacgatttct taaataacga accaaccaat ttccatccca tggactat |
| | >......................GLP-1......................>> |
| | e g q a a k e f i a w l v k g r |
| | >>..mTf..>> |
| | v p d |

After annealing and ligation, the clones were sequenced to confirm correct insertion. This vector was designated pREX0094. The cassette was cut out of pREX0094 with NotI and sub-cloned into NotI cut yeast vector, pSAC35, to make PREX0100.

This plasmid was then electroporated into the host Saccharomyces yeast strains and transformants selected for leucine prototrophy on minimal media plates. Expression was determined by growth in liquid minimal media and analysis of supernatant by SDS-PAGE, western blot, and ELISA.

GLP-1/mTf and H-GLP-1/mTf were expressed and purified from fermentation cultures, grown under standard conditions by cation exchange and anion exchange chromatography.

Dipeptidylpeptidase-IV Treatment

Equimolar concentrations of GLP-1/mTf and H-GLP/1-mTF (2 μM) were treated with recombinant human DPP-IV (1 μg/μL, R&D Systems) in a solution of 25 mM Tris-Cl (pH 8.0). Control reactions excluding DPP-IV were set-up in parallel for each fusion protein. The digests were incubated at room temperature for 2 hours, at which time the reactions were diluted 20-fold in Krebs-Ringer buffer (Biosource International) supplemented with 1 mM IBMX (Calbiochem).

Cyclic AMP Stimulation Assay

Tissue culture plates (24-well) were seeded with CHO-GLP1R cells at a density of 1×10⁵ cells per/well in RPMI/10% FBS medium one day prior to treatment. The next day the cells appeared uniformly distributed with an approximate confluency of 60-80 percent. One day after seeding the culture plates the cells were washed twice with Krebs-Ringer buffer (KRB) followed by incubation in KRB for 1 hr at 37° C. to lower the intracellular levels of cAMP. This was followed by incubation for 10 minutes in KRB/IBMX to inhibit intracellular enzymes that break down cAMP. Dilutions of each test compound were prepared in KRB/IBMX and triplicate wells of CHO-GLP1R cells were treated with 0.15 ml of test compound per well for exactly 50 minutes at 37° C. The treatment was halted by washing the cultures two times with ice-cold phosphate-buffered saline. Lysates were prepared by the addition of 0.2 ml lysis buffer 1B (Amersham Biosciences cAMP Biotrak EIA kit) for 10 minutes at room temperature, then 100 μl of each cell extract was then assayed to determine the cAMP concentration using the cAMP Biotrak Enzyme Immunoassay System (Amersham Biosciences) according to kit instructions.

H-GLP-1/mTf was found to be more resistant to DPP-IV than GLP-1/mTf

### Example 3

### Modified GLP-1/mTf for the Treatment of Diabetes

In this Example, modified GLP-1/mTf of the present invention is used as a therapeutic agent to treat diabetes. Modified GLP-1/mTf is administered to Zucker rats, a standard animal model for type II diabetes. Zucker rats have abnormally high blood glucose levels. It has been shown that treatment of these animals with GLP-1 induces insulin secretion and reduces blood glucose.

Zucker rats are fasted overnight and then treated with H-GLP-1 or H-GLP-1 fused to transferrin (H-GLP-1/mTf). Thirty minutes after subcutaneous injection of H-GLP-1 or H-GLP-1/mTf, the animals are subjected to a Glucose Tolerance Test (GTT). For this test, fasted animals are fed glucose solution (1.5 mg/g body weight), and the blood glucose is measured at appropriate time intervals. Soon after the glucose administration, the blood glucose level of the untreated animals rises and slowly drops towards the base line while the animals which are injected with H-GLP-1 or H-GLP-1/mTf show faster normalization of blood glucose level due to the insulinotropic effect of the GLP-1.

In a further experiment, modified H-GLP-1 or H-GLP-1/mTf is used to normalize the high fasting glucose of the Zucker rats without glucose administration. While the blood glucose levels remain high in the untreated animals, a significant drop is seen in the H-GLP-1 or modified H-GLP-1/mTf treated animals.

### Example 4

### Modified Glucagon Having Dipeptidyl-peptidase IV Protection

This Example describes modified glucagon molecules protected from DPPIV activity.

The following peptides are synthesized using standard solid phase Fmoc chemistry and purified by reverse phase HPLC using a C18 column and quantitated by absorbance at 220 nm. The purified peptides are analyzed by mass spectrometry (MALDI-TOF):

Glucagon

(SEQ ID NO: 35)

NH₂-His-Ser-Gln-Gly-Thr-Phe-Thr-Ser-Asp-Tyr-Ser-
Lys-Tyr-Leu-Asp-Ser-Arg-Arg-Ala-Gln-Asp-Phe-Val-
Gln-Trp-Leu-Met-Asn-Thr-COOH
H-Glucagon

(SEQ ID NO: 108)

NH₂-His-His-Ser-Gln-Gly-Thr-Phe-Thr-Ser-Asp-Tyr-
Ser-Lys-Val-Leu-Asp-Ser-Arg-Arg-Ala-Gln-Asp-Phe-
Val-Gln-Trp-Leu-Met-Asn-Thr-COOH

The peptides are pre-treated with DPP-IV as described above and then assayed for the ability to activate the glucagon receptor using a recombinant cell line expressing a cloned glucagon receptor.

### Example 5

### Modified GIP Having Dipeptidyl-peptidase IV Protection

This Example provides modified GIP molecules protected from DPPIV activity.

The following peptides are synthesized using standard solid phase Fmoc chemistry and purified by reverse phase HPLC using a C18 column and quantitated by absorbance at 220 nm. The purified peptides are analysed by mass spectrometry (MALDI-TOF):

GIP

(SEQ ID NO: 31)

NH₂-Tyr-Ala-Gly-Gly-Thr-Phe-Ile-Ser-Asp-Tyr-Ser-
Ile-Ala-Met-Asp-Lys-Ile-His-Gln-Gln-Asp-Phe-Val-
Asn-Trp-Leu-Leu-Ala-Gln-Lys-Gly-Lys-Lys-Asn-Asp-
Trp-Lys-His-Asn-Ile-Thr-Gln-COOH
Y-GIP

(SEQ ID NO: 109)

NH₂-Tyr-Tyr-Ala-Glu-Gly-Thr-Phe-Ile-Ser-Asp-Tyr-
Ser-Ile-Ala-Met-Asp-Lys-Ile-His-Gln-Gln-Asp-Phe-
Val-Asn-Trp-Leu-Leu-Ala-Gln-Lys-Gly-Lys-Lys-Asn-
Asp-Trp-Lys-His-Asn-Ile-Thr-Gln-COOH

The peptides are pre-treated with DPP-IV as described above and then assayed for the ability to activate the GIP receptor using a recombinant cell line expressing a cloned GIP receptor.

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents, and patent applications that are identified in this patent application are incorporated by reference in their entirety.

1. A polypeptide molecule modified to contain at least one additional amino acid at the N-terminal end that substantially protects the polypeptide molecule from dipeptidyl peptidase cleavage; wherein the modified polypeptide substantially retains polypeptide activity.

2. The polypeptide of claim 1, wherein the polypeptide is substantially protected from dipeptidyl peptidase IV cleavage.

3. The polypeptide of claim 1, wherein the modification substantially reduces the sensitivity of the polypeptide molecule to dipeptidyl peptidase cleavage.

4. A polypeptide of claim 1, wherein the polypeptide is modified to contain between one and five additional amino acids at its N-terminus.

5. A polypeptide of claim 1, wherein the polypeptide before modification comprises an N-terminal sequence X-Pro-Y or X-Ala-Y.

6. A polypeptide of claim 1, wherein the polypeptide before modification comprises an N-terminal sequence X-Ser-Y and X-Gly-Y.

7. A polypeptide of claim 1, wherein the additional amino acid is of the same class as the native N-terminal amino acid of the polypeptide before modification.

8. A polypeptide of claim 1, wherein the additional amino acid is identical to the native N-terminal amino acid of the polypeptide before modification.

9. A polypeptide of claim 1, wherein the modification reduces dipeptidyl-peptidase cleavage by at least about 30% compared to the polypeptide before modification.

10. A polypeptide of claim 1, wherein the modification reduces dipeptidyl-peptidase cleavage by at least about 50% compared to the polypeptide before modification.

11. A polypeptide of claim 1, wherein the modification reduces dipeptidyl-peptidase cleavage by at least about 70% compared to the polypeptide before modification.

12. A polypeptide of claim 1, wherein the modification reduces dipeptidyl-peptidase cleavage by at least about 90% compared to the polypeptide before modification.

13. A polypeptide of claim 1, wherein the modified polypeptide retains at least about 10% of its activity compared to the polypeptide before modification.

14. A polypeptide of claim 1, wherein the modified polypeptide retains at least about 30% of its activity compared to the polypeptide before modification.

15. A polypeptide of claim 1, wherein the modified polypeptide retains at least about 50% of its activity compared to the polypeptide before modification.

16. A polypeptide of claim 1, wherein the modified polypeptide retains at least about 70% of its activity compared to the polypeptide before modification.

17. A polypeptide of claim 1, wherein the modified polypeptide retains at least about 90% of its activity compared to the polypeptide before modification.

18. A polypeptide of claim 1, wherein the modification retains at least about 10% of its potency compared to the polypeptide before modification.

19. A polypeptide of claim 1, wherein the modification retains at least about 30% of its potency compared to the polypeptide before modification.

20. A polypeptide of claim 1, wherein the modification retains at least about 50% of its potency compared to the polypeptide before modification.

21. A polypeptide of claim 1, wherein the modification retains at least about 70% of its potency compared to the polypeptide before modification.

22. A polypeptide of claim 1, wherein the modification retains at least about 90% of its potency compared to the polypeptide before modification.

23. A polypeptide of claim 1, wherein the modification has increased potency compared to the polypeptide before modification.

24. A polypeptide of claim 1, wherein the polypeptide is a peptide hormone, chemokine or a neuropeptide.

25. A polypeptide of claim 14, wherein the polypeptide is selected from the group consisting of GLP-1, GLP-2, Exendin-3, Exendin-4, GIP, glucagon, neuropeptide Y, endomorphin, peptide YY, growth hormone-releasing hormone, gastric inhibitory polypeptide, RANTES, stromal cell-derived factor, eotaxin, macrophage-derived chemokine, substance P, and beta-casomorphins.

26. A polypeptide of claim 25, wherein the polypeptide is a GLP-1 polypeptide.

27. A polypeptide of claim 26, wherein the GLP-1 polypeptide is selected from the group consisting of GLP-1 (7-34), GLP-1 (7-35), GLP-1 (7-36) and GLP-1 (7-37).

28. A polypeptide of claim 26, wherein the N-terminal end of the GLP-1 polypeptide is selected from the group consisting of: His-His-Ala-Glu (SEQ ID NO: 82); His-His-Gly-Glu (SEQ ID NO: 83); His-His-Ser-Glu (SEQ ID NO: 84); Gly-His-Ala-Glu (SEQ ID NO: 85); Gly-His-Gly-Glu (SEQ ID NO: 86); Gly-His-Ser-Glu (SEQ ID NO: 87); and His-X-Ala-Glu, His-X-Gly-Glu, and His-X-Ser-Glu, wherein X is any amino acid.

29. A polypeptide of claim 28, wherein the N-terminal end of the GLP-1 polypeptide is His-His-Ala-Glu.

30. A polypeptide of claim 27, wherein the GLP-1 polypeptide sequence comprises: His-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly (SEQ ID NO: 88) or His-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (amino acids 1-31 of SEQ ID NO: 88).

31. A polypeptide of claim 27, wherein the GLP-1 polypeptide is GLP-1(7-36^{amide}).

32. A polypeptide of claim 27, wherein the GLP-1 polypeptide sequence comprises: His-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-X-Gly-Arg-Gly (SEQ ID NO: 89), wherein X is an amino acid other than lysine or is the D form of lysine; or His-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-X-Gly-Arg (amino acids 1-31 of SEQ ID NO: 89), wherein X is an amino acid other than lysine or is the D form of lysine.

33. A polypeptide of claim 28, wherein the His residue at the amino terminus has been chemically modified.

34. A polypeptide of claim 26, wherein the polypeptide is an analog of a GLP-1 polypeptide.

35. A polypeptide of claim 32, wherein X is selected from the group consisting of glutamine, alanine and asparagine.

36. A polypeptide of claim 1, wherein the polypeptide is fused to a second polypeptide.

37. A polypeptide of claim 36, wherein the second polypeptide is selected from the group comprising transferrin, lactotransferrin, melanotransferrin, and hybrids thereof.

38. A polypeptide of claim 36, wherein the second polypeptide is a modified transferrin.

39. A polypeptide of claim 37, wherein the transferrin polypeptide exhibits reduce glycosylation.

40. A polypeptide of claim 37, wherein the transferrin polypeptide is selected from the group consisting a single transferrin N domain, a single transferrin C domain, a transferrin N and C domain, two transferrin N domains and two transferrin C domains.

41. A polypeptide of claim 36, wherein the second polypeptide is selected from the group consisting of transferrin, immunoglobulin, an antibody Fc domain.

42. A polypeptide of claim 36, wherein the second polypeptide is a plasma protein.

43. A polypeptide of claim 1, wherein the polypeptide has been modified to extend the serum half-life when compared to the unmodified polypeptide.

44. A polypeptide of claim 43, wherein the polypeptide is pegylated.

45. A polypeptide of claim 44, wherein the polypeptide is conjugated to a heterologous molecule.

46. A polypeptide of claim 43, wherein the polypeptide is attached to a fatty acid derivative.

47. A polypeptide of claim 43, wherein the polypeptide contains a reactive group for binding to thiol.

48. An isolated nucleic acid molecule encoding a polypeptide of claim 1.

49. A composition comprising the nucleic acid of claim 48 and a carrier.

50. A vector comprising a nucleic acid molecule of claim 48.

51. A host cell comprising a vector of claim 50.

52. A host cell comprising a nucleic acid molecule of claim 48.

53. A method of expressing a polypeptide encoded by the nucleic acid of claim 48 in vivo comprising introducing the nucleic acid of claim 48 into an in vivo cell and allowing the cell to express the encoded polypeptide.

54. A method of treating a disease or condition in a patient, comprising administering an effective amount of a polypeptide of claim 1.

55. A method of claim 54, wherein the disease is a metabolic disease.

56. A method of claim 55, wherein the disease is type II diabetes.

57. A method of claim 55, wherein the polypeptide is a GLP-1 polypeptide.

58. A method of reducing the dipeptidyl-peptidase sensitivity of a polypeptide, comprising adding at least one additional amino acid at the N-terminal end that substantially protects the polypeptide molecule from dipeptidyl peptidase cleavage.

59. A method of claim 58, wherein the polypeptide is modified to contain between one and five additional amino acids at its N-terminus.

60. A method of claim 58, wherein the polypeptide before modification comprises an N-terminal sequences X-Pro-Y or X-Ala-Y.

61. A method of claim 58, wherein the polypeptide before modification comprises an N-terminal sequences X-Ser-Y or X-Ser-Y.

62. A method of claim 58, wherein the additional amino acid is of the same class as the native N-terminal amino acid of the polypeptide before modification.

63. A method of claim 58, wherein the additional amino acid is identical to the native N-terminal amino acid of the polypeptide before modification.

64. A method of claim 58, wherein the modification reduces dipeptidyl-peptidase cleavage by at least about 30% compared to the polypeptide before modification.

65. A method of claim 58, wherein the modification reduces dipeptidyl-peptidase cleavage by at least about 50% compared to the polypeptide before modification.

66. A method of claim 58, wherein the modification reduces dipeptidyl-peptidase cleavage by at least about 70% compared to the polypeptide before modification.

67. A method of claim 58, wherein the modification reduces dipeptidyl-peptidase cleavage by at least about 90% compared to the polypeptide before modification.

68. A method of claim 58, wherein the polypeptide is a peptide hormone, chemokine or a neuropeptide.

69. A method of claim 68, wherein the polypeptide is selected from the group consisting of GLP-1, GLP-2, GIP, glucagon, neuropeptide Y, endomorphin, peptide YY, growth hormone-releasing hormone, gastric inhibitory polypeptide, RANTES, stromal cell-derived factor, eotaxin, macrophage-derived chemokine, substance P, and beta-casomorphins.

70. A method of claim 69, wherein the polypeptide is a GLP-1 polypeptide.

71. A polypeptide of claim 32, wherein X is Arg.

72. A polypeptide of claim 36, wherein there is a linker between the polypeptide and the second polypeptide.

73. A method of claim 55, wherein the metabolic disease is diabetes.

74. A method of claim 55, wherein the metabolic disease is obesity.


SEQUENCE LISTING (119 sequences)

SEQ ID NO: 1 - protein, 5 aa, Homo sapiens
APVRS

SEQ ID NO: 2 - protein, 5 aa, Homo sapiens
APTSS

SEQ ID NO: 3 - protein, 5 aa, Homo sapiens
IPTEI

SEQ ID NO: 4 - protein, 5 aa, Homo sapiens
VPPGE

SEQ ID NO: 5 - protein, 5 aa, Homo sapiens
SPGQG

SEQ ID NO: 6 - protein, 5 aa, Homo sapiens
SPGPV

SEQ ID NO: 7 - protein, 5 aa, Homo sapiens
KPRLL

SEQ ID NO: 8 - protein, 5 aa, Homo sapiens
GPVSA

SEQ ID NO: 9 - protein, 5 aa, Homo sapiens
APARS

SEQ ID NO: 10 - protein, 5 aa, Homo sapiens
TPLGP

SEQ ID NO: 11 - protein, 5 aa, Homo sapiens
APPRL

SEQ ID NO: 12 - protein, 5 aa, Homo sapiens
FPTIP

SEQ ID NO: 13 - protein, 5 aa, Homo sapiens
VPLSR

SEQ ID NO: 14 - protein, 5 aa, Homo sapiens
MPITR

SEQ ID NO: 15 - protein, 5 aa, Homo sapiens
MPVER

SEQ ID NO: 16 - protein, 5 aa, Homo sapiens
GPETL

SEQ ID NO: 17 - protein, 5 aa, Homo sapiens
APLAT

SEQ ID NO: 18 - protein, 5 aa, Homo sapiens
MPMFI

SEQ ID NO: 19 - protein, 5 aa, Homo sapiens
EPDAI

SEQ ID NO: 20 - protein, 5 aa, Homo sapiens
YPSKP

SEQ ID NO: 21 - protein, 5 aa, Homo sapiens
APLEP

SEQ ID NO: 22 - protein, 5 aa, Homo sapiens
YPIKP

SEQ ID NO: 23 - protein, 5 aa, Homo sapiens
LPICP

SEQ ID NO: 24 - protein, 5 aa, Homo sapiens
SPYSS

SEQ ID NO: 25 - protein, 5 aa, Homo sapiens
RPKPQ

SEQ ID NO: 26 - protein, 5 aa, Homo sapiens
SPAPP

SEQ ID NO: 27 - protein, 5 aa, Homo sapiens
MPGMI

SEQ ID NO: 28 - protein, 5 aa, Homo sapiens
MPSCS

SEQ ID NO: 29 - protein, 5 aa, Homo sapiens
LPGVL

SEQ ID NO: 30 - protein, 5 aa, Homo sapiens
APMAE

SEQ ID NO: 31 - protein, 42 aa, Homo sapiens
YAEGTFISDYSIAMDKIHQQDFVNWLLAQKGKKNDWKHNITQ

SEQ ID NO: 32 - protein, 31 aa, Homo sapiens
  MISC_FEATURE (1)..(31): X at position 31 = G or -NH2.
HAEGTFTSDVSSYLEGQAAKEFIAWLVKGRX

SEQ ID NO: 33 - protein, 33 aa, Homo sapiens
HADGSFSDEMNTILDNLAARDFINWLIQTKITD

SEQ ID NO: 34 - protein, 44 aa, Homo sapiens
YADAIFTNSYRKVLGQLSARKLLQDIMSRQQGESNQERGARARL

SEQ ID NO: 35 - protein, 29 aa, Homo sapiens
HSQGTFTSDYSKYLDSRRAQDFVQWLMNT

SEQ ID NO: 36 - protein, 27 aa, Homo sapiens
HADGVFTSDFSKLLGQLSAKKYLESLM

SEQ ID NO: 37 - protein, 103 aa, Homo sapiens
GPETLCGAELVDALQFVCGDRGFYFNKPTGYGSSSRRAPQTGIVDECCFRSCDLRRLEMYCAPLKPAKSARSVRAQRHTDMPKAQKEVHLKNASRGSAGNKTY

SEQ ID NO: 38 - protein, 9 aa, Homo sapiens
RPPGFSPFR

SEQ ID NO: 39 - protein, 11 aa, Homo sapiens
RPKPQQFFGLM

SEQ ID NO: 40 - protein, 22 aa, Homo sapiens
RPVKVYPNGAEDESAEAFPLEF

SEQ ID NO: 41 - protein, 36 aa, Homo sapiens
YPSKPDNPGEDAPAEDMARYYSALRHYINLITRQRY

SEQ ID NO: 42 - protein, 36 aa, Homo sapiens
YPIKPEAPGEDASPEELNRYYASLRHYLNLVTRQRY

SEQ ID NO: 43 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 44 - protein, 92 aa, Homo sapiens
APDVQDCPECTLQEDPFFSQPGAPILQCMGCCFSRAYPTPLRSKKTMLVQKNVTSESTCCVAKSYNRVTVMGGFKVEDHTACHCSTCYYHKS

SEQ ID NO: 45 - protein, 145 aa, Homo sapiens
SKEPLRPRCRPINATLAVEKEGCPVCITVNTTICAGYCPTMTRVLQGVLPALPQVVCNYRNVRFESIRLPGCPRGVNPVVSYAVALSCQCALCRRSTTDCGGPKDHPLTCDDPRFQDSSSSKAPPPSLPSPSRLPKPSDTPILPQ

SEQ ID NO: 46 - protein, 5 aa, Homo sapiens
APGPR

SEQ ID NO: 47 - protein, 27 aa, Homo sapiens
VPLPAGGGTVLTKMYPRGNHWAVGHLM

SEQ ID NO: 48 - protein, 133 aa, Homo sapiens [sequence omitted]

SEQ ID NO: 49 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 50 - protein, 4 aa, Homo sapiens
YPFF

SEQ ID NO: 51 - protein, 4 aa, Homo sapiens
YPLG

SEQ ID NO: 52 - protein, 58 aa, Homo sapiens [sequence omitted]

SEQ ID NO: 53 - protein, 68 aa, Homo sapiens
SPYSSDTTPCCFAYIARPLPRAHIKEYFYTSGKCSNPAVVFVTRKNRQVCANPEKKWVREYINSLEMS

SEQ ID NO: 54 - protein, 246 aa, Homo sapiens
NPLLILTFVAAALAAPFDDDDKIVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 55 - protein, 184 aa, Homo sapiens
GPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRV

SEQ ID NO: 56 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 57 - protein, 74 aa, Homo sapiens
GPASVPTTCCFNLANRKIPLQRLESYRRITSGKCPQKAVIFLTKLAKDICADPKKKYVQDSMKYLDQKSPTPKP

SEQ ID NO: 58 - protein, 74 aa, Homo sapiens
QPDAINAPVTCCYNFTNRKISVQRLASYRRITSSKCPKEAVIFKTIVAKEICADPKQKWVQDSMDHLDKQTQTP

SEQ ID NO: 59 - protein, 76 aa, Homo sapiens
QPDSVSIPITCCFNVINRKIPIQRLESYTRITNIQCPKEAVIFKTKRGKEVCADPKERWVRDSMKHLDQIFQNLKP

SEQ ID NO: 60 - protein, 76 aa, Homo sapiens
QPVGINTSTTCCYRFINKKIPKQRLESYRRTTSSHCPREAVIFKTKLDKEICADPTQKWVQDFMKHLDKKTQTPKL

SEQ ID NO: 61 - protein, 77 aa, Homo sapiens
GPVSAVLTELRCTCLRVTLRVNPKTIGKLQVFPAGPQCSKVEVVASLKNGKQVCLDPEAPFLKKVIQKILDSGNKKN

SEQ ID NO: 62 - protein, 68 aa, Homo sapiens
KPVSLSYRCPCRFFESHVARANVKHLKILNTPNCALQIVARLKNNNRQVCIDPKLKWIQEYLEKALNK

SEQ ID NO: 63 - protein, 72 aa, Homo sapiens
KPVSLSYRCPCRFFESHVARANVKHLKILNTPNCALQIVARLKNNNRQVCIDPKLKWIQEYLEKALNKRFKM

SEQ ID NO: 64 - protein, 69 aa, Homo sapiens
GPYGANMEDSVCCRDYVRYRLPLRVVKHFYWTSDSCPRPGVVLLTFRDKEICADPRVPWVKMILNKLSQ

SEQ ID NO: 65 - protein, 7 aa, Homo sapiens
YPFVEPI

SEQ ID NO: 66 - protein, 95 aa, Homo sapiens
APGPRGIIINLENGELCMNSAQCKSNCCQHSSALGLARCTSMASENSECSVKTLYGIYYKCPCERGLTCEGDKTIVGSITNTNFGICHDAGRSKQ

SEQ ID NO: 67 - protein, 8 aa, Homo sapiens
HSDAVFTD

SEQ ID NO: 68 - protein, 6 aa, Homo sapiens
HSDGIF

SEQ ID NO: 69 - protein, 8 aa, Homo sapiens
HSQGTFTS

SEQ ID NO: 70 - protein, 8 aa, Homo sapiens
FPTIPLSR

SEQ ID NO: 71 - protein, 8 aa, Homo sapiens
HSDGTFTS

SEQ ID NO: 72 - protein, 31 aa, artificial
  substituted GLP-1
  MISC_FEATURE (1)..(31): X at position 2 = V or S; X at position 3 = Gln, D-Gln or Asp; X at position 10 = T or G; X at position 12 = K or A.
HXXGTFTSDXSXYLEGQAAKEFIAWLVKGRG

SEQ ID NO: 73 - protein, 31 aa, artificial
  substituted GLP-1
  MISC_FEATURE (1)..(31): X at position 20 = G, S, C, T, N, Q, Y, A, V, I, L, M, F, A, d-Lys or K; X at position 28 = G, S, C, T, N, Q, Y, A, V, I, L, M, F, A, d-Lys or K; X at position 30 = G, S, C, T, N, Q, Y, A, V, I, L, M, F, K, R, d-Arg or -OH.
  MISC_FEATURE (1)..(31): X at position 29 = G or -OH; X at position 31 = G or -OH.
HAEGTFTSDVSSYLEGQAAXEFIAWLVXXXX

SEQ ID NO: 74 - protein, 31 aa, artificial
  substituted GLP-1
  MISC_FEATURE (1)..(31): X at position 25 = oxidation-resistant amino acid; X at position 29 = G or -OH; X at position 30 = R or -OH; X at position 31 = G or -OH.
HAEGTFTSDVSSYLEGQAAKEFIAXLVXXXX

SEQ ID NO: 75 - protein, 31 aa, artificial
  substituted GLP-1
  MISC_FEATURE (1)..(31): X at position 10 = Y or V; X at position 12 = K or S; X at position 15 = D or E; X at position 16 = S or G; X at position 17 = R or Q; X at position 18 = R or A; X at position 20 = Q or K.
  MISC_FEATURE (1)..(31): X at position 29 = G or -OH; X at position 30 = R or -OH; X at position 31 = G or -OH.
HAEGTFTSDXSXYLXXXXAXEFIAWLVXXXX

SEQ ID NO: 76 - protein, 31 aa, artificial
  substituted GLP-1
  MISC_FEATURE (1)..(31): X at position 2 = G or S or C or A; X at position 3 = D or G or S or C or T or N or Q or Y or A or V or I or L or M or F or E; X at position 4 = S or C or T or N or Q or Y or A or V or I or L or or F or G; X at position 9 = E or D.
  MISC_FEATURE (1)..(31): X at position 29 = G or -OH; X at position 30 = R or -OH; X at position 31 = G or -OH.
HXXXTFTSXVSSYLEGQAAKEFIAWLVXXXX

SEQ ID NO: 77 - protein, 31 aa, artificial
  substituted GLP-1
  MISC_FEATURE (1)..(31): X at position 1 = G or S or C or T or N or Q or Y or A or V or I or L or M or F or d-His or alkylated His or acylated His; X at position 29 = G or -OH; X at position 30 = R or -OH; X at position 31 = G or -OH.
XAEGTFTSDVSSYLEGQAAKEFIAWLVXXXX

SEQ ID NO: 78 - protein, 31 aa, artificial
  substituted GLP-1
  MISC_FEATURE (1)..(31): X at position 1 = l- or d-His, desamino-His, 2-amino-His, beta-hydroxy-His, homohistidine, alpha-fluoromethyl- His, and alpha-methyl-His; X at position 2 = A, G, V, T, I or alpha-methyl-Ala; X at positions  15 and 21 = E, Q, A, T, S or G.
  MISC_FEATURE (1)..(31): X at position 31 = Gly or NH2.
XXEGTFTSDVSSYLXGQAAKXFIAWLVKGRX

SEQ ID NO: 79 - protein, 39 aa, Homo sapiens
HSDGTFTSDLSKQMEEEAVRLFIEWLKNGGPSSGAPPPS

SEQ ID NO: 80 - protein, 39 aa, Homo sapiens
HGEGTFTSDLSKQMEEEAVRLFIEWLKNGGPSSGAPPPS

SEQ ID NO: 81 - protein, 31 aa, artificial
  modified exendin-4
HGEGTFTSDLSKQMEEAVRLFIEWLKNGGPY

SEQ ID NO: 82 - protein, 4 aa, artificial
  modified GLP-1 N-terminus
HHAE

SEQ ID NO: 83 - protein, 4 aa, artificial
  modified GLP-1 N-terminus
HHGE

SEQ ID NO: 84 - protein, 4 aa, artificial
  modified GLP-1 N-terminus
HHSE

SEQ ID NO: 85 - protein, 4 aa, artificial
  modified GLP-1 N-terminus
GHAE

SEQ ID NO: 86 - protein, 4 aa, artificial
  modified GLP-1 N-terminus
GHGE

SEQ ID NO: 87 - protein, 4 aa, artificial
  modified GLP-1 N-terminus
GHSE

SEQ ID NO: 88 - protein, 32 aa, artificial
  modified GLP-1
HHAEGTFTSDVSSYLEGQAAKEFIAWLVKGRG

SEQ ID NO: 89 - protein, 32 aa, artificial
  modified GLP-1
  MISC_FEATURE (1)..(31): X = an amino acid other than l- or d-Lys
HHAEGTFTSDVSSYLEGQAAKEFIAWLVXGRG

SEQ ID NO: 90 - protein, 30 aa, artificial
  A8G modified GLP-1
HGEGTFTSDVSSYLEGQAAKEFIAWLVKGR

SEQ ID NO: 91 - protein, 31 aa, artificial
  modified GLP-1 with additional N-terminal His
HHAEGTFTSDVSSYLEGQAAKEFIAWLVKGR

SEQ ID NO: 92 - protein, 31 aa, artificial
  modified GLP-1 with additional N-terminal His and A8G
HHGEGTFTSDVSSYLEGQAAKEFIAWLVKGR

SEQ ID NO: 93 - protein, 32 aa, artificial
  modified GLP-1 with 2 additional N-terminal His residues
HHHAEGTFTSDVSSYLEGQAAKEFIAWLVKGR

SEQ ID NO: 94 - protein, 31 aa, artificial
  modified GLP-1 with additional N-terminal Gly
GHAEGTFTSDVSSYLEGQAAKEFIAWLVKGR

SEQ ID NO: 95 - protein, 40 aa, artificial
  modified exendin-4 with additional N-terminal His
HHGEGTFTSDLSKQMEEEAVRLFIEWLKNGGPSSGAPPPS

SEQ ID NO: 96 - DNA, 40 bp, artificial
  primer for constructing modified GLP-1- modified Tf fusion protein
ttcccatacaaacttaagagtccaattagcttcatcgcca

SEQ ID NO: 97 - DNA, 60 bp, artificial
  primer for constructing modified GLP-1- modified Tf fusion protein
ggtttagcttgtttttttattggcgatgaagctaattggactcttaagtttgtatgggaa

SEQ ID NO: 98 - DNA, 60 bp, artificial
  primer for constructing modified GLP-1- modified Tf fusion protein
ataaaaaaacaagctaaacctaattctaacaagcaaagatgaggctcgccgtgggagccc

SEQ ID NO: 99 - DNA, 60 bp, artificial
  primer for constructing modified GLP-1- modified Tf fusion protein
caggacggcgcagaccagcagggctcccacggcgagcctcatctttgcttgttagaatta

SEQ ID NO: 100 - DNA, 70 bp, artificial
  primer for constructing modified GLP-1- modified Tf fusion protein
tgctggtctgcgccgtcctggggctgtgtctggcgcatgctgaaggtacttttacttctgatgtttcttc

SEQ ID NO: 101 - DNA, 80 bp, artificial
  primer for constructing modified GLP-1- modified Tf fusion protein
aattctttagcagcttgaccttccaaataagaagaaacatcagaagtaaaagtaccttcagcatgcgccagacacagccc

SEQ ID NO: 102 - DNA, 70 bp, artificial
  primer for constructing modified GLP-1- modified Tf fusion protein
ttatttggaaggtcaagctgctaaagaatttattgcttggttggttaaaggtagggtacctgataaaact

SEQ ID NO: 103 - DNA, 40 bp, artificial
  primer for constructing modified GLP-1- modified Tf fusion protein
agttttatcaggtaccctacctttaaccaaccaagcaata

SEQ ID NO: 104 - DNA, 300 bp, artificial
  sequence encoding modified GLP-1-modified Tf fusion protein
  CDS (109)..(300)
ccaatgttacgtcccgttatattggagttcttcccatacaaacttaagagtccaattagcttcatcgccaataaaaaaacaagctaaacctaattctaacaagcaaagatgaggctcgccgtgggagccctgctggtctgcgccgtcctggggctgtgtctggcgcatgctgaaggtacttttacttctgatgtttcttcttatttggaaggtcaagctgctaaagaatttattgcttggttggttaaaggtagggtacctgataaaactgtgagatggtgtgcagtgtcggagcatgag

SEQ ID NO: 105 - protein, 64 aa, artificial
  sequence encoding modified GLP-1-modified Tf fusion protein
MRLAVGALLVCAVLGLCLAHAEGTFTSDVSSYLEGQAAKEFIAWLVKGRVPDKTVRWCAVSEHE

SEQ ID NO: 106 - DNA, 25 bp, artificial
  primer for constructing plasmid with natural Tf secretion signal
ctgtgtctggcgcatcatgctgaag

SEQ ID NO: 107 - DNA, 25 bp, artificial
  primer for constructing plasmid with natural Tf secretion signal
cttcagcatgatgcgccagacacag

SEQ ID NO: 108 - protein, 30 aa, artificial
  modified glucagon with additional N-terminal His
HHSQGTFTSDYSKVLDSRRAQDFVQWLMNT

SEQ ID NO: 109 - protein, 43 aa, artificial
  modified GIP with additional N-terminal Tyr
YYAEGTFISDYSIAMDKIHQQDFVNWLLAQKGKKNDWKHNITQ

SEQ ID NO: 110 - protein, 5 aa, artificial
  dipeptidyl peptidase serine protease motif
GWSYG

SEQ ID NO: 111 - protein, 6 aa, artificial
  transferrin secretion signal sequence
RSLDKR

SEQ ID NO: 112 - protein, 6 aa, artificial
  transferrin secretion signal sequence
RSLDRR

SEQ ID NO: 113 - protein, 6 aa, artificial
  transferrin secretion signal sequence
RSLEKR

SEQ ID NO: 114 - protein, 6 aa, artificial
  transferrin secretion signal sequence
RSLERR

SEQ ID NO: 115 - protein, 4 aa, artificial
  DPP-resistant N-terminus
HHAE

SEQ ID NO: 116 - protein, 4 aa, artificial
  DPP-resistant N-terminus
GHAE

SEQ ID NO: 117 - DNA, 90 bp, artificial
  DNA sequence encoding GLP-1(7-36)
catgctgaaggtacttttacttctgatgtttcttcttatttggaaggtcaagctgctaaagaatttattgcttggttggttaaaggtaga

SEQ ID NO: 118 - DNA, 118 bp, artificial
  site of pREX0052 with GLP-1 insert
  CDS (1)..(117)
aggtctctagagaaaaggcatgctgaaggtacttttacttctgatgtttcttcttatttggaaggtcaagctgctaaagaatttattgcttggttggttaaaggtagggtacctgata

SEQ ID NO: 119 - protein, 39 aa, artificial
  site of pREX0052 with GLP-1 insert
RSLEKRHAEGTFTSDVSSYLEGQAAKEFIAWLVKGRVPD